(19) United States
(12) Patent Application Publication
(10) Pub. No.: US 2008/0033466 A1
(43) Pub. Date: Feb. 7, 2008

(54) Surgical cutter with exchangeable cutter blades

(75) Inventors: Robert L. Assell, Wilmington, NC (US); Eugene A. Dickhudt, Lino Lakes, MN (US); Thomas M. Womble, Leland, NC (US)

Correspondence Address: THE ECLIPSE GROUP, 10605 BALBOA BLVD., SUITE 300, GRANADA HILLS, CA, 91344, US

(73) Assignee: TranS1 Inc., Wilmington, NC (US)

(21) Appl. No.: 11/891,686

(22) Filed: Aug. 10, 2007

Related U.S. Application Data
(63) Continuation-in-part of application No. 11/712,548, filed on Feb. 28, 2007.
(63) Continuation-in-part of application No. 11/712,241, filed on Feb. 28, 2007.
(60) Provisional application No. 60/837,201, filed on Aug. 10, 2006.
(60) Provisional application No. 60/778,035, filed on Feb. 28, 2006.

(51) Int. Cl. A61B 17/32

(52) U.S. Cl. 606/171; 606/167

(57) ABSTRACT

Cutters for disrupting tissue include a cutter body and replaceable cutter blades. The cutter blades may be placed in one of three positions (sheathed for transport, unsheathed for use, and sufficiently exposed for blade exchange) by the relative movement of a cutter sheath with respect to the engagement zone between the cutter blade and the cutter body. The relative movement of the cutter sheath may be limited to an operational range to preclude inadvertent exposure of the cutter blade to prevent unintended disengagement from the cutter body. Various sheath limiters are disclosed that selectively limit movement of the cutter sheath to an operational range or allow extraordinary movement to allow blade exchange. In some instances other intermediate stops may be used to further limit the relative motion. The relative motion could be imparted by the movement of a cutter rod (rather than the movement of the cutter sheath) and thus limited by a rod limiter. Cutter blades with different attributes (such as throw length, cutter blade angle, type and location of blade edges) are adapted to achieve different objectives.

This application builds upon a series of applications filed on behalf of assignee. In particular this application claims priority to and incorporates by reference co-pending and commonly assigned U.S. Provisional Application No. 60/837,201 filed Aug. 10, 2006 for Method and Reusable Apparatus for Tissue Excision. This application claims priority to and incorporates by reference, and extends the innovative work in the area of manipulating material in the spine described in two co-pending and commonly assigned U.S. patent application Ser. No. 11/712,548 filed Feb. 28, 2007 for Cutter for Preparing Intervertebral Disc Space and application Ser. No. 11/712,241 for Specialized Cutter Blades for Preparing Intervertebral Disc Space, both of which in turned claim priority to U.S. Provisional Patent Application No. 60/778,035 for Method and Apparatus for Tissue Manipulation and Extraction filed Feb. 28, 2006. This application claims priority and incorporates by reference the 60/778,035 application.

This application incorporates by reference but does not claim priority to U.S. patent application Ser. No. 10/972,077 for Method and Apparatus for Manipulating Material in the Spine filed Oct. 22, 2004 and subsequently published as United States Patent Application No. US 2005/0149034 A1. This application incorporates by reference various applications claimed as priority documents by the '077 application specifically: U.S. Provisional Patent Application No. 60/513,899, filed on Oct. 23, 2003, and U.S. patent application Ser. No. 10/309,416, filed on Dec. 3, 2002 (now U.S. Pat. No. 6,921,403), which is a continuation-in-part of U.S. patent application Ser. No. 10/125,771, filed on Apr. 18, 2002 (now U.S. Pat. No. 6,899,716), which is a continuation-in-part of U.S. patent application Ser. No. 09/848,556, filed on May 3, 2001, (now U.S. Pat. No. 7,014,633) which is a continuation-in-part of U.S. patent application Ser. No. 09/782,583, filed on Feb. 13, 2001 (now U.S. Pat. No. 6,558,390), which claims priority to U.S. Provisional Patent Application No. 60/182,748, filed on Feb. 16, 2000. U.S. patent application Ser. No. 09/782,534 teaches various types of techniques for using cutting tools for removing disc material and preparation of spinal treatment sites that comprise a spinal disc, for example, a method of removing at least a portion of the nucleus through an anterior tract axial bore while leaving the annulus fibrosus intact.

While a number of applications have been incorporated by reference to provide additional detail it should be noted that these other applications (including those that have subsequently issued as patents) were written at an earlier time and had a different focus from the present application. Thus, to the extent that the teachings or use of terminology differ in any of these incorporated applications from the present application, the present application controls.

### BACKGROUND

1. Field of the Invention

This disclosure relates generally to improved cutters and methods for disrupting tissue including bone as part of a therapeutic procedure including preparing treatment sites within the spine, such at the intervertebral space between two adjacent vertebral bodies for subsequent therapeutic procedures including therapies such as spinal fusion or procedures where fusion of the two adjacent vertebral bodies is not desired such as therapies for the implantation of motion preservation devices into the spine.

2. Overview

The present disclosure is an extension of work in a series of patent applications (some now issued patents) with a common assignee. Much of the work is described in great detail in the many applications referenced above and incorporated by reference into this application. Accordingly, the background provided here does not repeat all of the detail provided in the earlier applications, but instead highlights how the present disclosure adds to this body of work.

The spinal column is a complex system of bone segments (vertebral bodies and other bone segments) which are in most cases separated from one another by discs in the intervertebral spaces (sacral vertebrae are an exception). In the context of the present disclosure, a “motion segment” includes adjacent vertebrae, i.e., an inferior (caudal; proximal with respect to a trans sacral access) and a superior (cephalad; distal with respect to a trans sacral access) vertebral body, and the intervertebral disc space separating said two vertebral bodies, whether denucleated space or with intact or damaged spinal discs. Unless previously fused (or damaged), each motion segment contributes to the overall flexibility of the spine contributes to the overall ability of the spine to flex to provide support for the movement of the trunk and head.

The vertebrae of the spinal cord are conventionally subdivided into several sections. Moving from the head to the tailbone, the sections are cervical, thoracic, lumbar, sacral, and coccygeal. The individual vertebral bodies within the sections are identified by number starting at the vertebral body closest to the head. The trans-sacral approach is well suited for access to vertebral bodies in the lumbar section and the sacral section. As the various vertebral bodies in the sacral section are usually fused together in adults, it is sufficient and perhaps more descriptive to merely refer to the sacrum rather than the individual sacral components.

It is useful to set forth some of the standard medical vocabulary before getting into a more detailed discussion of the background of the present disclosure. When referencing tools including cutters, distal would be the end intended for insertion into the access channel and proximal refers to the other end, generally the end closer to the handle for the tool, or to the operator

The individual motion segments within the spinal columns allow movement within constrained limits and provide protection for the spinal cord. The discs are important to cushion and distribute the large forces that pass through the spinal column as a person walks, bends, lifts, or otherwise moves. Unfortunately, for a number of reasons referenced below, for some people, one or more discs in the spinal column will not operate as intended. The reasons for disc problems range from a congenital defect, disease, injury, or degeneration attributable to aging. Often when the discs are not operating properly, the gap between adjacent vertebral bodies is reduced and this causes additional problems including pain.

A range of therapies have been developed to alleviate the pain associated with disc problems. One class of solutions is to remove the failed disc and then fuse the two adjacent vertebral bodies together with a permanent but inflexible spacing, also referred to as static stabilization. Fusing one section together ends the ability to flex in that motion segment. While the loss of the normal physiologic disc function for a motion segment through fusion of a motion segment may be better than continuing to suffer from the pain, it would be better to alleviate the pain and yet retain all or much of the normal performance of a healthy motion segment.

Another class of therapies attempts to repair the disc so that it resumes operation with the intended intervertebral spacing and mechanical properties. One type of repair is the replacement of the original damaged disc with a prosthetic disc. This type of therapy is called by different names such as dynamic stabilization or spinal motion preservation.

Additional details on the operation of the spine and on specific therapies to treat motion segments are provided in the various applications and patents that are referenced above. For purposes of this application, it is sufficient to note that as part of the provision of therapy, that tissue may need to be disrupted and removed. Tissue in the context of the spine includes material in the intervertebral disc, and also vertebrae endplates, and bone within the interior of the vertebrae. Other medical procedures, including procedures that do not treat the spine may use cutters or cutter bodies made in accordance with this disclosure to disrupt other tissue or analogous tissue in other parts of the body.

U.S. patent application Ser. No. 11/712,548 filed Feb. 28, 2007 for Cutter for Preparing Intervertebral Disc Space and application Ser. No. 11/712,241 for Specialized Cutter Blades for Preparing Intervertebral Disc Space describe specific cutter blades that are adapted for particular spinal procedures. The details are incorporated by reference and do not need to be described in great detail here. Suffice it to say that blades used to prepare a site for fusion may be adapted to promote scraping of the vertebral endplates that are adjacent to the intervertebral disc space. In contrast, blades that are being used for a procedure that does not wish to promote bone growth between the adjacent vertebrae would tend to protect the endplates from scraping. Scraping is to be avoided as scraping promotes bleeding and bleeding promotes bone growth.

Design of cutter blades includes considerations in many cases of the efficiency with which the cutter blade prepares the contents of the nucleus for removal by cutting (slicing, tearing, or some combination of the two). It is generally desirable to allow a surgeon to work quickly and efficiently to reduce the time of surgery which has benefits in reducing the length of time that a patient is kept under anesthesia and as an added benefit also reduces the use of expensive resources such as the surgical team and the surgical suite.

Certain procedures may benefit from a series of cutter blades of different properties including progressively longer throw lengths and perhaps a variety of blade angles in order to efficiently disrupt the tissue within a region. There are advantages to having a cutter (which is a cutter body and a cutter blade) that allows a cutter blade to be removed and replaced with another cutter blade. The replacement blade may be identical to the blade it replaces. Alternatively, the replacement blade may have a different blade angle or blade throw to allow the surgeon to efficiently reach tissue that could not be efficiently reached with the previous cutter blade. In another instance, the replacement blade may have a similar blade angle or blade throw as to the one it replaces but the cutting action of the cutter blade may be different.

A procedure that needs only three cutter blades might be handled by three cutters preloaded with the cutter blades anticipated to be used in the procedure. However as each cutter body must be either sterilized or discarded after use with a given patient, there is a cost associated with having a large number of cutter bodies come in contact with a particular patient. For a procedure using many cutter blades or potentially needing to use a cutter blade configuration that was not anticipated, there are advantages to being able to remove a cutter blade from a cutter and replace it with a different cutter blade. Ideally, such a replacement process would be something that a gloved assistant could do quickly within a sterile field of an operating room

Thus, a process that may use a dozen cutter blades may be performed with only a single cutter body. It is anticipated that usually two or three cutter bodies would be used so that the surgeon does not need to wait while cutter blades are removed and replaced.

A wide variety of efforts has been proposed or attempted in the prior art, in an effort to relieve back pain and restore physiological function. Notwithstanding these efforts, there remains a need for methods and tools for accessing and preparing an intervertebral motion segment for subsequent therapeutic procedures, which can be accomplished in a minimally invasive manner. In particular, there are disclosed herein reusable cutter bodies, disposable cutter blades, and kits of components for particular therapeutic procedures. One such procedure is axial access to and preparation of disc spaces and extraction of nucleus pulposus material. The devices and kits offer the surgeon enhanced, real-time procedural options and flexibility, with respect to use of a plurality of specialized cutter blades designed to accommodate patients' anatomical variability, as well as applicability for multiple clinical indications. The clinician and the patient benefit from the use and reuse of a single cutter body and cutter blade combinations capable of enabling different tissue removal actions ranging from radial scraping to shearing excision, via an inherent ability to more safely and effectively detach and switch blade configurations in the operating room, in real-time.

### SUMMARY OF THE DISCLOSURE

This disclosure describes a series of cutters for disrupting tissue. The cutters include a cutter body and replaceable cutter blades. The cutter body has a cutter rod and the cutter blades may be reversibly attached to the cutter body such as by engagement with a post on the distal end of the cutter body in a slot dimensioned to receive a portion of the cutter blade in a cutter blade engagement zone. The cutter body has a sheath that is substantially coaxial with the cutter rod. The sheath may include a sheath liner, which may be replaced in order to prolong the useful life of the remainder of the sheath. The sheath liner may be made from a material that has a lower coefficient of friction with respect to the cutter blades than does the material used for making the sheath.

The cutter blades engaged in a cutter body may be placed in one of at least three positions (sheathed for transport, unsheathed for use, and sufficiently exposed for blade exchange) by the relative movement of a cutter sheath with respect to the engagement zone between the cutter blade and the cutter body. The relative movement of the cutter sheath may be limited to an operational range to preclude inadvertent exposure of the cutter blade to prevent unintended disengagement from the cutter body. Various sheath limiters are disclosed that selectively limit movement of the cutter sheath to an operational range or allow extraordinary movement to allow blade exchange. In some instances other intermediate stops may be used to further limit the relative motion such as to partially sheathe a cutter blade to alter the blade angle of the cutter blade. The sheath limiter may have a position that allows for the removal of the sheath from the cutter body as part of a disassembly process.

The relative motion could be imparted by the movement of a cutter rod (rather than the movement of the cutter sheath) and thus limited by a rod limiter. Cutter blades with different attributes (such as throw length, cutter blade angle, closed loop versus thin blade, type and location of blade edges) are adapted to achieve different objectives.

The concepts disclosed could be implemented in a range of cutter body/cutter blade combinations that could be used for a range of therapeutic actions including performance of a nucleectomy via a trans-sacral access channel.

This summary is meant to provide an introduction to the concepts that are disclosed within the specification without being an exhaustive list of the many teachings and variations upon those teachings that are provided in the extended discussion within this disclosure. Thus, the contents of this summary should not be used to limit the scope of the claims that follow.

Other systems, methods, features and advantages of the disclosed teachings will be or will become apparent to one with skill in the art upon examination of the following figures and detailed description. It is intended that all such additional systems, methods, features and advantages be included within the scope of and be protected by the accompanying claims.

### BRIEF DESCRIPTION OF THE FIGURES

The disclosure can be better understood with reference to the following figures. The components in the figures are not necessarily to scale, emphasis instead being placed upon illustrating the principles disclosed. Moreover, in the figures, like reference numerals designate corresponding parts throughout the different views.

FIGS. 1A-1C illustrates an anterior trans-sacral axial access method of creating an axial channel in the spine.

FIG. 2 shows a cutter assembly inserted into an axial channel with the cutter blade in an unsheathed position.

FIG. 3 addresses the concept of using a series of cutter blades of different throw lengths within an intervertebral disc space.

FIG. 4 depicts a different alignment between the axial channel 212 and the endplates of the two vertebral bodies.

FIG. 5 shows blade arms for cutter blades with a angles of 45 degrees, 90 degrees, and 135 degrees with respect to a longitudinal portion of the cutter blade.

FIGS. 6A-6C show three views of a closed loop cutter blade with cutting edges on the inside perimeter of the closed loop cutting blade.

FIGS. 7A-7D show four views of a closed loop cutter blade with cutting edges on the outside perimeter of the closed loop cutting blade.

FIGS. 8A-8D show four views of a low profile cutter blade.

FIG. 9 shows the major components of a cutter.

FIG. 10A-10C shows a cutter blade being added to a cutter body and retained by the cutter sheath.

FIGS. 11A-11D provide a side cross section of the proximal end of a cutter to display various combinations of positions of sheath limiter 704 and sheath grip 648.

FIG. 12A-12D provide four views of the sheath limiter 704.

FIG. 13A-13D provide four views of a sheath grip 648.

FIGS. 14A-14C provide three views of a snap lock 908 sheath liner.

FIG. 15 provides a perspective view from the distal end of a threaded sheath liner.

FIG. 16 is an exploded diagram of the proximal end of a cutter rod and handle assembly 1004 showing only a portion of the cutter rod 624.

FIG. 17 is an enlarged side view of dual threaded thumb screw 1024.

FIG. 18 shows a portion of a cross section of cutter rod and handle sub-assembly 1004 in the operational position.

FIG. 19 shows a portion of a cross section of a cutter rod and handle sub-assembly 1004 in the blade exchange position, one of the extraordinary as opposed to operational positions.

FIG. 20 shows an exploded view of a cutter handle and sheath limiter from a cutter body 1100.

FIG. 21 shows the sheath limiter 1120 in the operational position and the cutter body 1100 positioned to have a cutter blade 453 (not shown here) unsheathed but retained so that the cutter may be used to disrupt tissue.

FIG. 22 shows the sheath limiter 1120 in the operational position with the operational bore 1146 engaged and the sheath limiter 1120 precluded from slotted movement and the sheath grip at the forward stop.

FIG. 23 shows the sheath limiter 1120 outside of the operational position and in the position for a blade change.

FIGS. 24A-24B illustrate a sheath limiter 1200 with an intermediate stop 1204.

### DETAILED DESCRIPTION

While the inventive cutters described below may be used in other surgical procedures, it is useful in context to describe how these cutters could be adapted for use in a trans-sacral approach. As noted above there are many advantages associated with a minimally invasive, low trauma trans-sacral axial approach. The trans-sacral axial approach (described and disclosed in commonly assigned U.S. Pat. Nos. 6,558,386; 6,558,390; 6,575,979; 6,921,403; 7,014,633, and 7,087,058) has a number of advantages over other routes for delivery of therapeutic devices to motion segments but there are logistical challenges to the preparation of an intervertebral disc space via an axial access channel. The process of addressing these challenges impacts certain aspects of the cutters intended for use in this manner.

Trans-Sacral Axial Access.

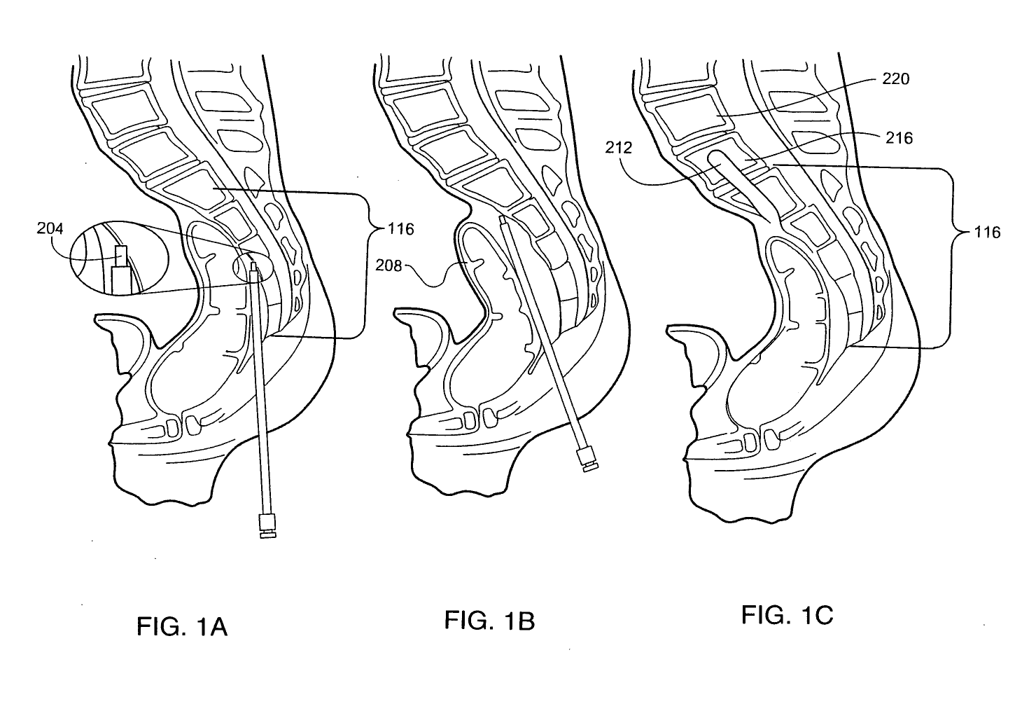

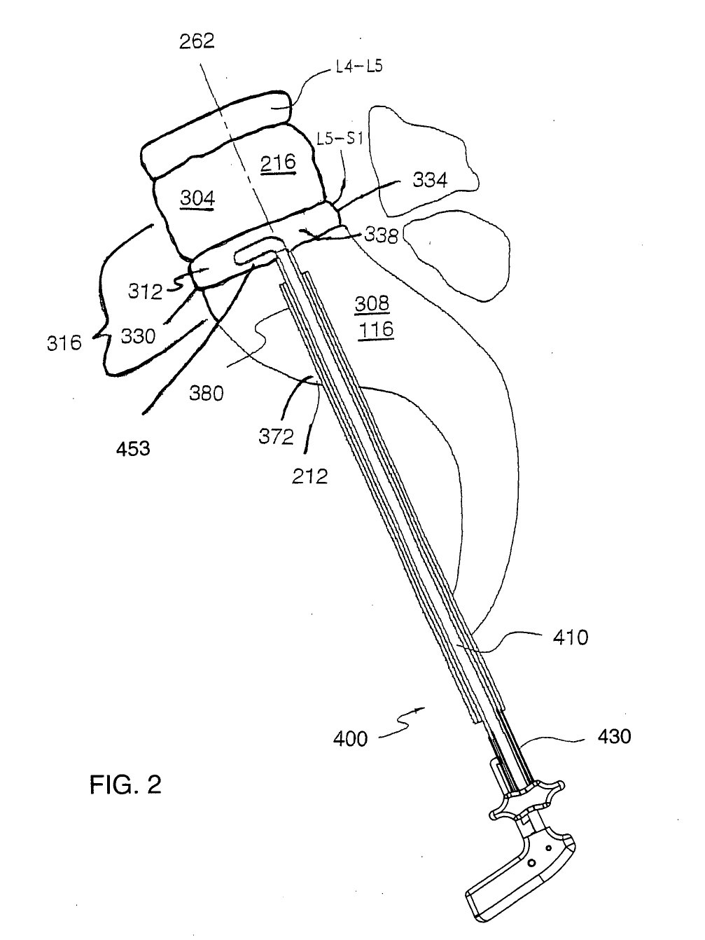

The trans-sacral axial access method illustrated in FIGS. 1A-1C, eliminates the need for muscular dissection and other invasive steps associated with traditional spinal surgery while allowing for the design and deployment of new and improved instruments and therapeutic interventions, including stabilization, motion preservation, and fixation devices/fusion systems across a progression-of-treatment in intervention.

FIGS. 1A-1C provides an introductory overview of the process with FIG. 1A and FIG. 1B showing the process of “walking” a blunt tip stylet 204 up the anterior face of the sacrum 116 to the desired position on the sacrum 116 while monitored one or more fluoroscopes (not shown). This process moves the bowel 208 out of the way so that a straight path is established for the subsequent steps. FIG. 1C illustrates a representative trans-sacral axial channel 212 established through the sacrum 116, the L5/S1 intervertebral space, and into the L5 vertebra 216. If therapy is being provided to the L4/L5 motion segment then the channel would continue through the L5 vertebra 216 through the L4/L5 intervertebral space, and into the L4 vertebra 220.

The discussion of FIGS. 1A-1C is provided to provide context for the present disclosure. Previous applications (some now issued as United States patents) with common assignee have included a description of an alternative access method that is a posterior trans-sacral axial spinal approach rather than an anterior trans-sacral axial spinal approach. (See e.g. U.S. Pat. No. 6,558,386 for Axial Spinal Implant and Method and Apparatus for Implanting an Axial Spinal Implant Within the Vertebrae of the Spine as this patent describes the anterior trans-sacral axial approach illustrated in FIGS. 1A-1C and is incorporated by reference in its entirety.)

Referring to FIG. 2, a cutter 400 is inserted through the axially aligned anterior tract 372 defined by the lumen of the dilator sheath 380 and the axial channel 212 which is difficult to see as the dilator sheath 380 substantially fills the axial channel 212 as it passes through the sacrum 116. (One of skill in the art will appreciate that the axial channel 212 may be extended axially by a sequence of steps so that the length of an axial channel may include additional vertebral bodies or intervertebral disc spaces). One of skill in the art will appreciate that due to anatomical differences the axial channel for some therapies may circumvent the sacrum and may enter through another portion of the spine.

As shown in FIG. 2, motion segment 316 that includes the proximal vertebra 308 (the sacrum 116), the intervertebral space 312 (in this case the L5-S1 space with disc 330, annulus fibrosus 334 and nucleus 338), the distal vertebra 304 (in this case L5 216). The cutter 400 comprises a cutting blade (e.g., cutter blade 453 which refers collectively to any blade configuration) which is remotely manipulable. The manipulations of the cutter blade 453 may include sheathing the cutter blade 453 by extending a cutter sheath 430 over the cutter blade 453 so that the maximum radius of the cutter 400 is reduced and the cutter assembly with the sheathed blade 453 may be advanced through the axial channel 212. The spring tension from the sheathed blade 453 pushing outward against the cutter sheath 430 (or sheath liner as discussed below) holds the cutter sheath 430 in place. After reaching the location where the cutter blade 453 is to be operated, the cutter blade 453 may be unsheathed. Even in the unsheathed position, the cutter blade 453 applies sufficient spring force against the interior of the cutter sheath or sheath liner to hold the cutter sheath 430 in position.

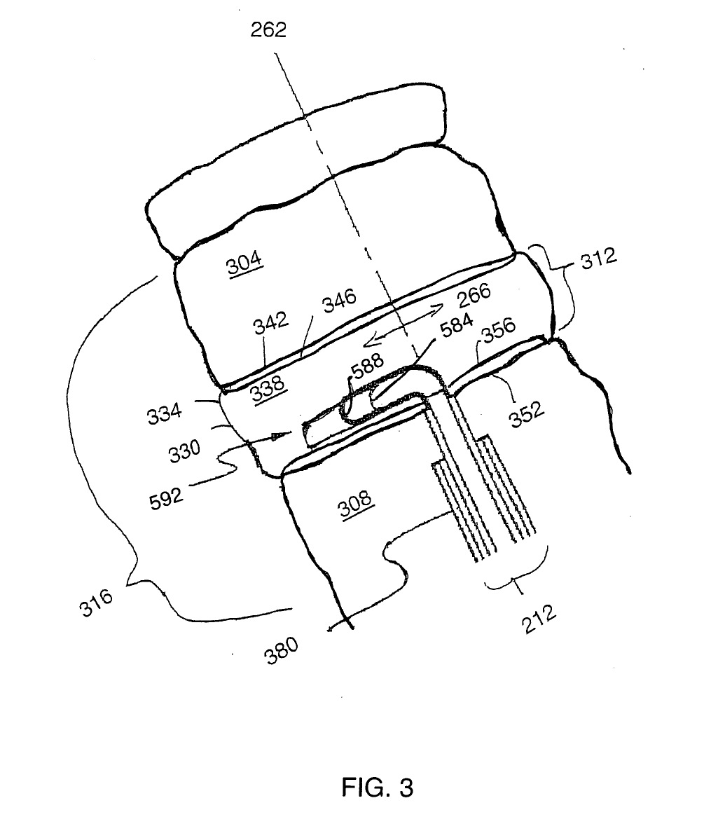

As shown in FIG. 3, the centerline 262 of the cutter 400 is close to the centerline of the axial channel 212 due to the fit of the dilator sheath 380 in the axial channel 212 and the fit of the cutter 400 within the dilator sheath 380. When the cutter blade 453 is unsheathed as shown in FIG. 3 the distal portion of the cutter blade 453 is substantially transverse to the centerline 262 of the cutter 400. The unsheathed cutter blade 453 is extended into the nucleus 338 of the spinal disc 330.

The cutter rod 410, cutter sheath 430 (shown in FIG. 2) and the handle components are preferably co-configured to enable the cutter blade 453 and the cutter rod 410 to which it is attached be able to be “pushed-pulled” so as to sheathe the cutter blade 453 which looks as if the cutter blade 453 is being withdrawn into the cutter sheath 430 when actually the cutter sheath 430 is moving away from the cutter handle to sheathe the cutter blade 453.

More specifically, the cutter blade edges(s) of the cutter blade 453 are covered by the advanced cutter sheath 430 for delivery into the intervertebral disc space 312. Once the cutter 400 is in position, the cutter blade 453 is unsheathed by withdrawing the cutter sheath 430 and rotated using the handle to cut or otherwise disrupt tissue within the intervertebral disc space 312. After completing the cutting task or when the cutter blade 453 needs replacement, the cutter blade 453 is again retracted into the cutter sheath 430 for removal of the cutter 400 from the axial channel 212.

Overview of Cutter Blades.

After this introduction to cutters, it is useful to discuss why a sequence of cutter blades may be used while preparing the interior of an intervertebral disc space 312. FIG. 3 shows a first example. In FIG. 3 a motion segment 316 including a distal vertebral body 304, an intervertebral disc space 312 (with a intervertebral disc 330 including an annulus fibrosus 334, and nucleus pulposus 338 and bounded by the endplates), and a proximal vertebral body 308 are shown. For purposes of this example, it is not important which vertebral bodies are involved beyond the need for them to be adjacent vertebral bodies.

FIG. 3 includes the endplate 342 of the distal vertebral body 304 and a representation of the layer of cartilage 346 located on the endplate 342 which defines one portion of the intervertebral disc space 312. Assuming the route of access is a trans-sacral axial access, from the point of reference of the intervertebral disc space 312, endplate 342 would be the superior endplate. Likewise FIG. 3 includes the endplate 352 of the proximal vertebral body 308 and a representation of the layer of cartilage 356 located on the endplate 352 which defines one portion of the intervertebral disc space 312. Assuming the route of access is a trans-sacral axial access, from the point of reference of the intervertebral disc space 312, endplate 352, would be the inferior endplate.

One of skill in the art will recognize that the inclusion of the cartilage layers 346 and 356 is for purposes of discussing the use of cutters and is not intended to be an anatomically correct and appropriately dimensioned representation of cartilage.

The position of the cutter within the intervertebral disc space may be visible to the surgeon under real-time fluoroscopic imaging (possibly both anterior/posterior and lateral imaging).

In order to illustrate a point, FIG. 3 includes representations of three different cutter blades 584, 588, and 592 of differing throw lengths. One of ordinary skill in the art will appreciate that one method for cutting the nucleus 338 would use a series of cutter blades (584, 588, 592, and possibly another longer blade) to gradually cut the nucleus 338. One of ordinary skill in the art will understand that these three blades of different throw lengths (sometime called reaches) would be used sequentially from shorter to longer and it is only for the point of illustration that three different blade lengths are shown simultaneously in FIG. 3. To provide context, the reach of a series of cutter blades used in a particular procedure may range from about 0.40 inches for a small cutter blade to about 0.70 inches for a large cutter blade. One of skill in the art will recognize that these ranges are illustrative and could be different. It will be understood that the optimum throw for cutter blades depends on several factors, including patient anatomy and axial entrance point into the disc space, as well as issues related to sagittal symmetry of the spinal disc. Moreover, for safety reasons, it may be desirable to limit the length of the cutter blade to preclude a throw that is too close to the disc edge, in other words to avoid making contact between the cutter blade and the annulus fibrosus 334 to preclude compromising the annulus fibrosus.

Note that the distal portions of the cutter blades 584, 588, and 592 when unsheathed are transverse to the centerline of the cutter 262 and parallel to the axis 266 that is perpendicular to cutter blade centerline 262. The cutter blades are also close to parallel to the endplates 342 and 352 and the layers of cartilage 346 and 356.

In this example, the successively longer cutter blades 584 588, and 592, could be rotated 360 degrees or more around the centerline 262. Some surgeons may prefer to work on one segment at a time by rotating the cutter handle a fraction of 360 degrees (perhaps approximately 90 degrees) then rotating the cutter handle in the opposite direction to return to the position occupied by the cutter. Thus, the process tends to proceed while working on radial quadrants. Sometimes this short movement is compared to the movement of windshield wipers on an automobile.

In addition to using a series of cutter blades with sequentially increasing throws, the surgeon will need to adjust the axial position of the cutter blade by sliding the cutter further into the patient and away from the surgeon or towards the distal portion of the motion segment so that the cutter blade move sequentially closer to the cartilage 346 on the endplate 342 on the distal vertebral body 304. The surgeon may opt to create a first space relatively close to the proximal vertebral body by using a sequence of cutters of increasing throws then repeating the process with the cutter extended further into the nucleus (and repeating the sequence of blades of increasing throws).

Alternatively, the surgeon may choose to use one or more cutters with a first throw to create a space approximating a cylinder that is substantially the height of the space between the two layers of cartilage and a radius approximately equal to a first blade throw. This process may involve the use of a radial cutter blade with a given throw length followed by one or more cutter blades at a different blade angle(s) (for example 45 degrees) but the same throw length. Once the cutting is complete for a given throw length, the surgeon moves to cutter blades of a longer throw length starting again with a radial cutter blade. This process may be repeated with cutter blades of increasing blade throws until the desired amount of space is created.

The nature of the therapeutic procedure and the patient anatomy will determine the maximum cutter blade throw length required. Certain procedures may tend to use a greater number of cutter blade throw lengths to make smaller incremental increases in throw length. Other procedures may simply use a small throw length then move to the maximum throw length needed to prepare the intervertebral disc space.

As the nucleus material is cut, the surgeon may periodically remove the cutter from the axial channel and use any appropriate tissue extractor tool. U.S. patent application Ser. No. 10/972,077 (referenced above) describes several retractable tissue extractors that may be used for this purpose.

U.S. patent application Ser. No. 10/972,077 (referenced above) noted that when preparing a intervertebral disc space for a fusion procedure, it can be advantageous to use cutters to scrape away the cartilaginous endplate and roughen the vascularized vertebral body so as to cause bleeding, which is desirable in order to facilitate bone growth and to promote fusion of the vertebral bodies of the relevant motion segment.

However, not all therapeutic procedures seek to obtain such bleeding to promote fusion. It is unavoidable to disturb the a portion of endplate 352 of the proximal vertebral body as the axial channel is created through the endplate 352 and it is likewise unavoidable to disturb a portion of the cartilage 356 in the immediate vicinity of the axial channel (likewise the endplate 342 and cartilage 346 of the distal vertebral body 304 if the axial channel 212 (FIG. 1C) is extended into the distal vertebral body 304). However, the unavoidable disturbance of a small portion of an endplate and cartilage does not remove the advantage within certain procedures of avoiding damage to other portions of the cartilage and endplate.

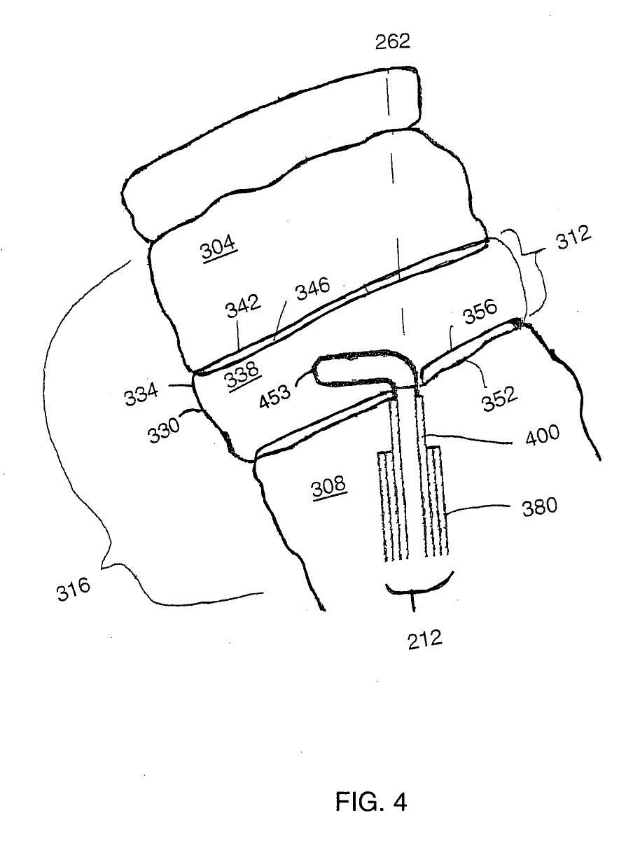

FIG. 4 depicts a different alignment between the axial channel 212 and the endplates of the two vertebral bodies. In FIG. 4, a cutter assembly 400 passed into and partially through a dilator sheath 380 in the axial channel 212 would have the cutter centerline 262 at an angle that is not close to perpendicular to the endplate 352 of the proximal vertebral body 308 or the endplate of the 342 of the distal vertebral body (the inferior and superior endplates of the intervertebral disc space 312).

A cutter blade 353 with an angle between the cutter rod 310 and the cutter blade 353 of approximately 90 degrees would be useful in cutting a portion of the nucleus, but would be less effective in removing other portions of the nucleus.

FIG. 4 is intended to highlight the need for cutter blades with blade angles other than 90 degrees. FIG. 4 is not intended as an indication of an optimal alignment of an axial channel for any particular therapeutic procedure. In actual medical procedures, while planning the placement of a axial channel, the surgeon will evaluate and select an alignment that provides for appropriate clearance from anatomic structures to allow for safe and effective implantation including effective anchoring within the relevant vertebral bodies.

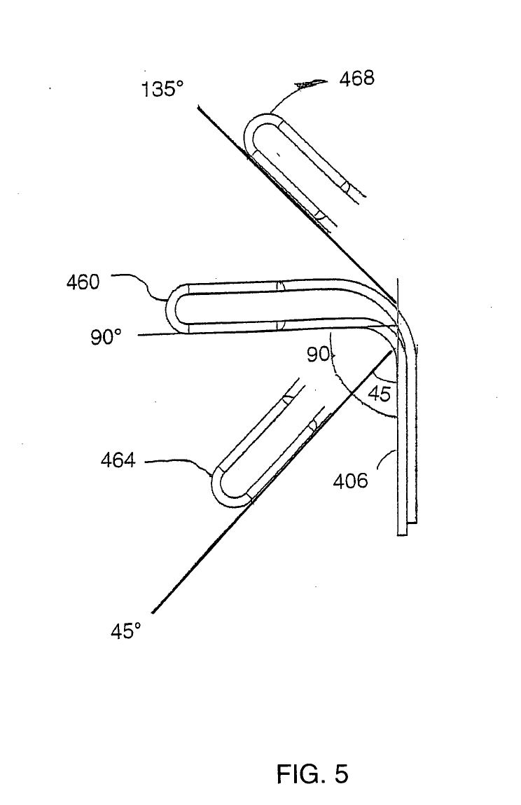

FIG. 5 illustrates a naming convention that is useful when discussing another attribute of cutter blades. In this case cutter blade 460 is a 90 degree cutter blade as there is a 90 degree angle (nominal) between the proximal side portion of the blade arm and the longitudinal portion 406 of the cutter blade 460. A portion of a 45 degree cutter blade 464 is shown with the more proximal portion of the portion of the cutter blade 464 at approximately 45 degrees with respect to the back of the longitudinal portion 406. While not shown here, an intermediate portion would connect the portion of the cutter blade 464 to a longitudinal portion 406.

Likewise a portion of a 135 degree cutter blade 468 is shown with the more proximal portion of the portion of the 135 degree cutter blade 468 at approximately 135 degrees with respect to the back of the longitudinal portion 406.

Note that as can be observed below the longitudinal portion 406 of a cutter blade 460 is going to be substantially parallel to the length of the cutter rod 410 and the cutter sheath 430, and the centerline axis of the cutter 262 so that these lines could be used for measuring the cutter blade angle.

One of skill in the art will recognize that to the extent that the cutter blades are produced in a finite number of nominal cutter blade angles, the actual measurement of the precise angle may deviate a few degrees (perhaps 5) from the nominal angle value. The actual angle may deviate over cycles of moving from the sheathed to the unsheathed position.

In many situations a set of cutter blades of various combinations of throw lengths and angles (such as 45 degree, 90 degree, and 135 degree) may be sufficient. Some surgeons may feel that they obtain adequate results for some therapies with using just 90 degree and 45 degree cutter blades. Other angles could be used, including angles that deviate less from 90 such as 60 and 120 degrees, or angles that deviate more from 90 degrees such as 25 and 155 degrees. Angles even closer to 90 degrees may be useful in some applications such as an angle in the vicinity of 105 degrees. Kits could include more than three angle values for the cutter blades. For example, a kit might include blades at 25, 45, 60, 90, 105, 120, 135 and 155 degree angles. With this range of blade angles, there is a wide variation of the extent to which the extended blades are transverse to the long axis of the cutter assembly, but in all these cases the cutter blades are significantly transverse to the long axis of the cutter assembly and to the longitudinal portions of the cutter blades. In addition to having a variety of blade angles and throw lengths, a kit may contain cutter blades of more than one type such as a mix of closed loop cutter blades and low profile cutter blades (both discussed below). The kit may contain cutter blades with different cutter blade edges, such as including serrated blades and non-serrated blades.

Some surgeons may work on a situation such as presented in FIG. 4 by initially using a short 90 degree cutter blade, then using progressively longer 90 degree cutter blades (one or more cutter blades with longer throws) to cut as much material within the intervertebral disc space 312 as can be safely handled using 90 degree cutter blades. Then the surgeon may want to work with a short 45 degree cutter blade then one or more longer 45 degree cutter blades to remove material that would be difficult to access using a 90 degree cutter blade. Finally, in some cases, the surgeon may opt to use a short 135 degree cutter blade followed by one or more longer 135 degree cutter blades to cut nucleus material that is difficult to access using either a 90 degree or a 45 degree cutter blade.

Details on Particular Types of Cutter Blades.

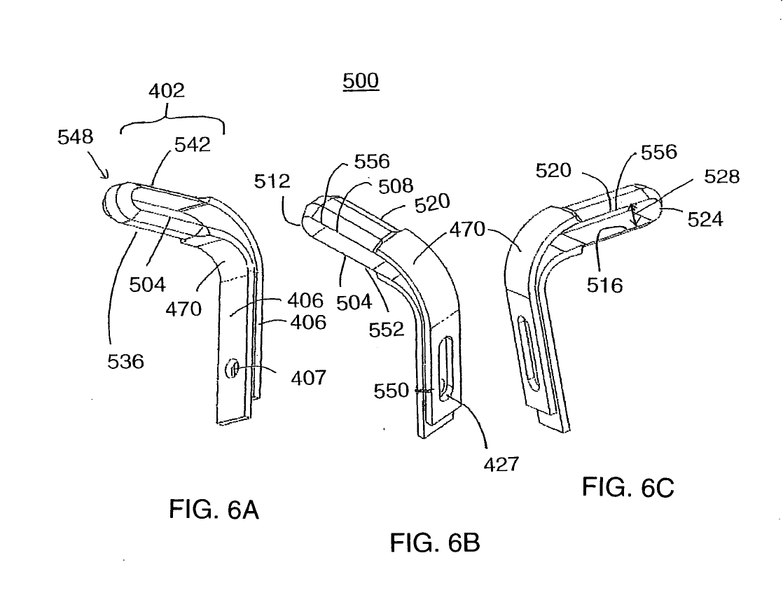

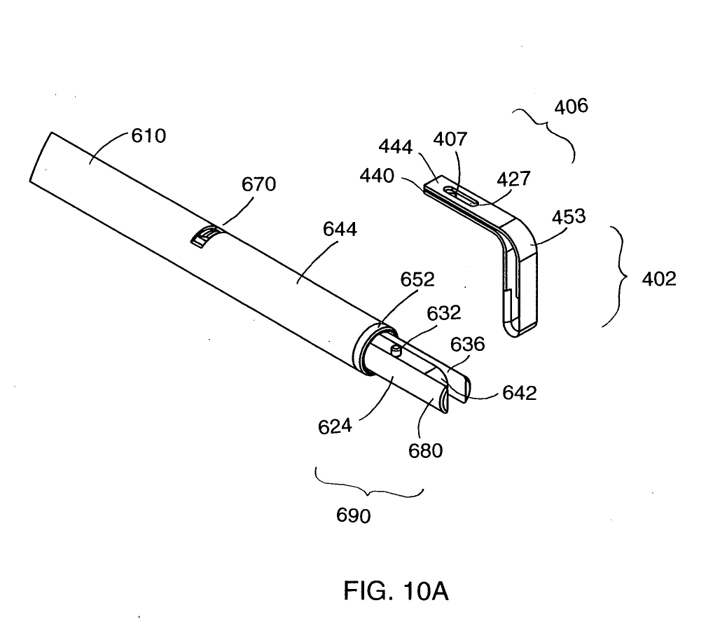

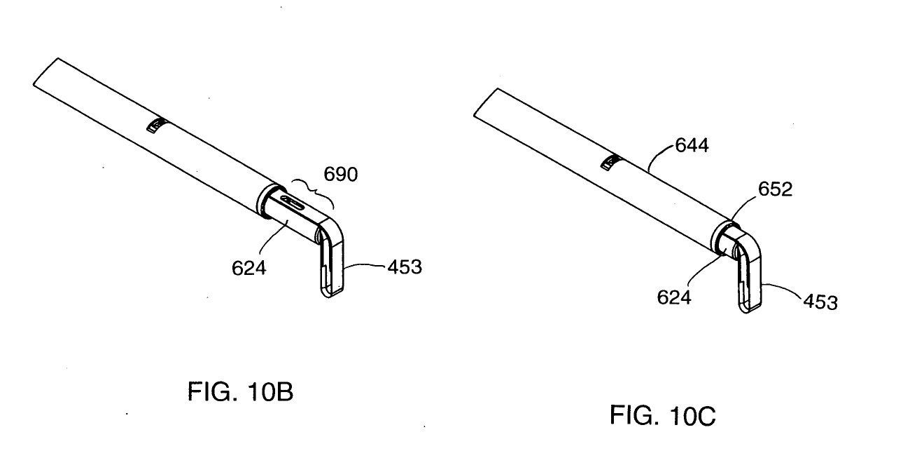

FIG. 6 shows three views of a particular cutter blade 500. Visible are the cutter blade hole 407 and the cutter blade slot 427. The cutter blade arm 402 is joined to the longitudinal portions 406 by a pair of transitional sections 470. While the precise position is not particularly relevant, in the area where the two transitional sections 470 meet the two longitudinal sections 406, the two ends of the cutter blade meet. This point of contact could be deemed place where the loop is closed. However, it may be simpler to call the loop closed at 550 which is placed at cutter blade hole 407 and the currently adjacent portion of cutter blade slot 427 as those two are joined when the cutter blade is attached to the cutter assembly at the blade shaft (See FIG. 10B)

The closed loop adds a layer of redundancy in that in the event of a break in cutter blade 500 while inserted into an intervertebral disc space, all portions of the cutter blade 500 will remain connected to the cutter rod through either the portion of the cutter blade with the slot 427 or the portion of the cutter blade with a hole 407. As all parts of the cutter blade are connected to the cutter rod even after a break in the cutter blade, the parts can be removed from the intervertebral disc space by prompt removal of the cutter assembly. The redundancy provides a safeguard against leaving a piece of a broken cutter blade in a patient upon withdrawal of the rest of the cutter blade which would create a need for a separate set of steps to remove the piece from within the patient.

Surgeons may note the break in the cutter blade either by a change in feel in the operation of the cutter or by a visible change in the cutter blade as indicated in the real-time fluoroscopic imaging.

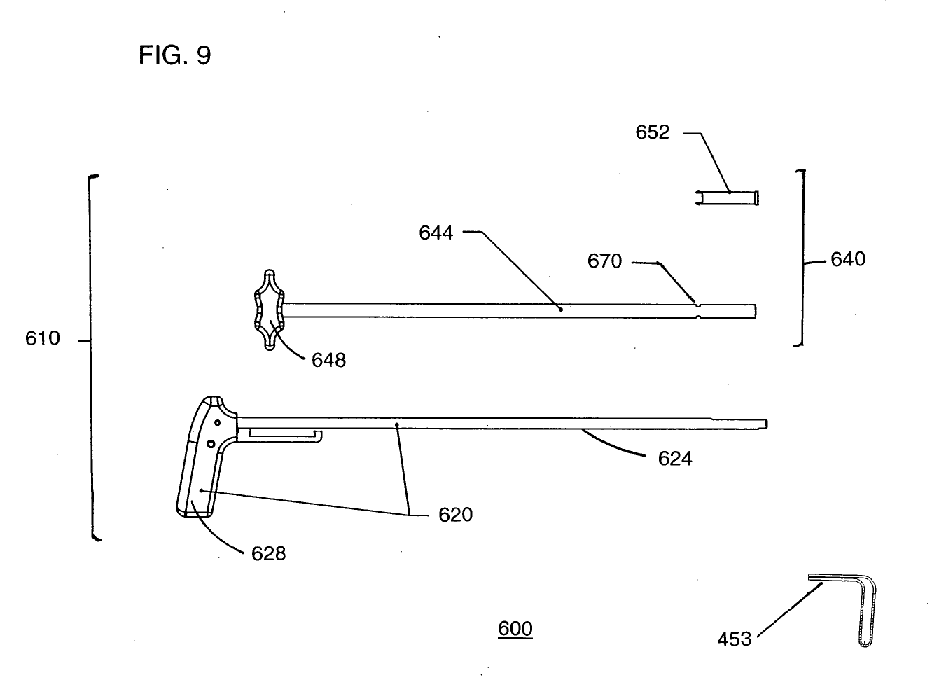

Cutter blade 500 can be said to have six different cutting edges 504, 508, 512, 516, 520, 524. Three cutting edges 504, 508, 512 on one side and three cutting edges 516, 520, 524 on the other side. Edges 504 and 516 are on the proximal portion 536 of the cutter blade 500, that is the portion of the cutter blade 500 that is closer to the cutter handle (628 in FIG. 9) than the other portion of the closed loop that is the distal portion 542 of the cutter blade 500. When inserted into the intervertebral disc space, the exterior of the proximal portion 536 will generally face the endplate on the proximal vertebral body (whether or not the proximal portion is parallel to the endplate). Edges 508 and 520 are on the distal portions 542 of the cutter blade 500. When inserted into the intervertebral disc space, the exterior of the distal portion 542 will generally face the endplate on the distal vertebral body (whether or not the distal portion 542 is parallel to the endplate). Edges 512 and 524 are on the tip 548 of the cutter blade 500 between the distal portion 542 and the proximal portion 536.

Note that the sides of a cutter blade are not necessarily flat. The sides (sometimes called faces) have features that are visible when looking at that side or face of the object (just as the indentations on one of the six faces of a single die from a pair of dice are visible when looking at that face or side of the die).

In each case, the cutting edges are on the inner perimeter 552 of the closed loop rather than on the outer perimeter 556 as the outer perimeter 556 might possibly contact the cartilage on an endplate. By recessing the cutting edges relative to the outer perimeter 556 of the closed loop, the cutter blade 500 is adapted to minimize trauma to either the cartilage 356 (FIG. 3) on the proximal endplate 352 (likely to be the inferior endplate when viewed in context of the intervertebral disc space 312) or the cartilage 346 (FIG. 3) on the distal endplate 342 (likely to be the superior endplate when viewed in the context of the intervertebral disc space 312). Although the cutter blade 500 has a nominal blade angle of 90 degrees, as illustrated in FIG. 4, it would not be impossible for such a cutter blade 500 to make contact with the cartilage on the superior endplate.

By having cutting edges on both sides of cutter blade 500, the surgeon may cut nucleus material while rotating the cutter blade in the clockwise direction and also while rotating the cutter blade in the counter-clockwise direction. (Clockwise and counterclockwise are dependent on orientation. One way of defining clockwise would be as viewed from the cutter while looking from proximal towards distal end of the cutter assembly. This would match the way the surgeon would view rotation of the cutter handle.

While being bidirectional is a useful feature, not all cutter blades must have cutting edges on both sides. Likewise, some cutter blades may have one type of cutting edge on one side and a second type of cutter blade on the second side. While it may be advantageous for some cutter blades to have blade edges on the tips of the cutter blade (such as blade edges 512 and 524 in FIG. 6), some cutter blades may not have a blade edge in the tip or may have a different blade edge type in the tip 548 than in the distal portion 542 and proximal portion 536.

The cutting blade 500 has a gap 528 within the closed loop that may allow material to pass through the gap while the cutter blade 500 is being rotated within the intervertebral disc space 312. This may add another aspect to the cutting action while reducing the resistance to the cutter blade 500 moving through the intervertebral disc space 312. Other cutter blades may have less of a gap between the distal and proximal portions or no gap at all. A cutter blade without a gap large enough to allow material to pass through the gap in the inside perimeter of the close loop receives benefit from the closed loop as noted above in that having the closed loop connected to the cutter rod provides two points of connection for the cutter blade and provides at least one point of connection from each part of the cutter blade to the cutter rod 410 in the event of a break in the cutter blade.

The cutter blade 500 may be described as having a reverse bevel to place the cutting edges away from the outer perimeter. Note that while the blade edges 504, 508, 512, 516, 520, and 524 on cutter blade 500 are recessed all the way to the inner perimeter 552 of the closed loop, other cutter blades seeking to avoid damaging cartilage or endplates may recess the blade edges to be away from the outer perimeter 556 of the closed loop but not all the way to the inner perimeter 552 of the closed loop. The blade edges may, for example, be midway between the outer perimeter 556 and the inner perimeter 552 and be sufficiently recessed to avoid damaging the cartilage.

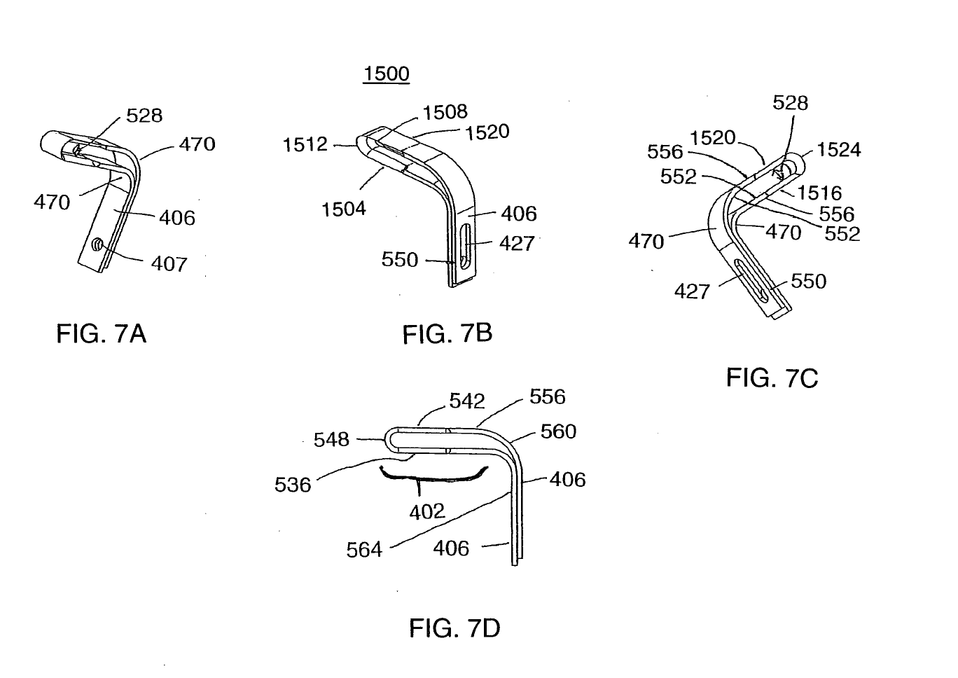

As noted above, it may cutter blades may be designed to promote abrasion of vertebral endplates rather than avoid abrasion as is the case for the cutter 500 in FIG. 6A-C. FIG. 7 shows a particular cutter 1500 that has blade edges on the outer perimeter 556 of the closed loop.

Cutter blade 1500 can be said to have six different cutting edges 1504, 1508, 1512, 1516, 1520, 1524. Three cutting edges 1504, 1508, 1512 on one side and three cutting edges 1516, 1520, 1524 on the other side. Edges 1504 and 1516 are on the proximal portion 536 of the blade arm 402 of the cutter blade 500, that is the portion of the blade arm that is closer to the handle 628 (FIG. 9) than the other portion of the closed loop that is the distal portion 542 of the blade arm 402.

When inserted into the intervertebral disc space, the exterior of the proximal portion 536 will generally face the endplate on the proximal vertebral body (whether or not the proximal portion is parallel to the endplate). Edges 1508 and 1520 are on the distal portion 542 of the blade arm 402. When inserted into the intervertebral disc space, the exterior of the distal portion 542 will generally face the endplate on the distal vertebral body (whether or not the distal portion 542 is parallel to the endplate). Edges 1512 and 1524 are on the tip 548 of the cutter blade 1500 between the distal portion 542 and the proximal portion 536 of the blade arm 402 and connecting the distal arm 560 and the proximal arm 564.

The cutting edges along the proximal portion 536 and the distal portion 542 of the blade arm 402 do not extend over the entire blade arm 402. As indicated in FIG. 3 it is contemplated that a series of cutter blades of increasing length will be used so that the cutter blade edges do not need to extend over the entire range that was previously cut by a previous cutter blade.

Note that the sides of a cutter blade are not necessarily flat. In each case, the six cutting edges are on the outer perimeter 556 of the closed loop rather than on the inside perimeter 552 as the outer perimeter 556 is the better choice for edge placement in order to contact the cartilage on an endplate. By placing the cutting edges on the outer perimeter 556 of the closed loop, the cutter blade 1500 is adapted to maximize the effectiveness of the cutter blade in cutting either the cartilage 356 (FIG. 3) on the proximal endplate 352 (likely to be the inferior endplate when viewed in context of the intervertebral disc space 312) or the cartilage 346 (FIG. 3) on the distal endplate 342 (likely to be the superior endplate when viewed in the context of the intervertebral disc space 312).

While there are many advantages to a closed loop cutter blade such as shown as cutter blade 500 or cutter blade 1500 discussed above, other cutter blades have advantages in certain situations.

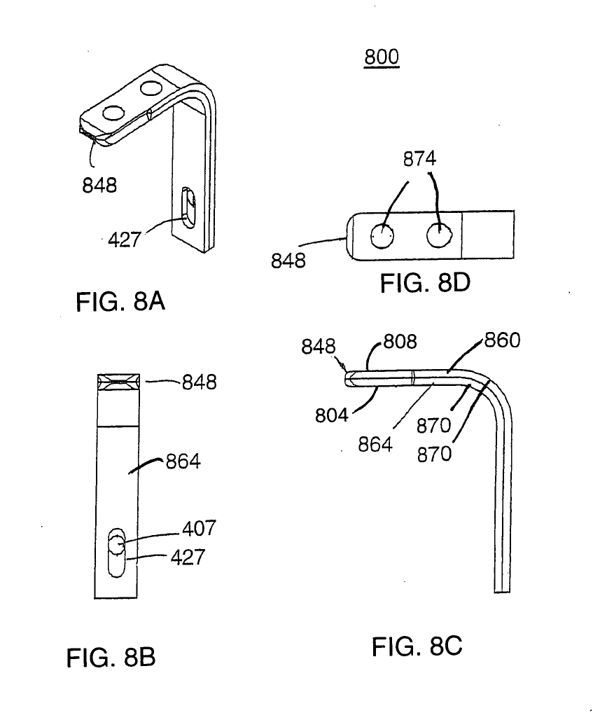

FIGS. 8A-D shows a low profile cutter blade 800 for use in situations such as a collapsed disc. The low profile cutter blade 800 has many features that are similar to the closed loop cutter blade 1500 discussed in connection with FIG. 7. However, unlike the closed loop cutter blades 500 or 1500, there is not a gap between the distal arm 860 and the proximal arm 864 in the vicinity of the blade edges 804 and 808. Thus the thickness of the cutter blade is on the order of magnitude of only 0.050 inches which is considerably less than found in the closed loop cutter blades such as cutter blade 500 or 1500.

FIG. 8A is a top perspective view of low profile cutter blade 800. As FIG. 8A shows the entire low profile cutter blade 800 it includes cutter blade slot 427. FIG. 8B, a front view of low profile cutter blade 800 shows cutter blade slot 427 that is on the proximal arm 864 and visible through the cutter blade slot 427 is the cutter blade hole 407 that is on distal arm 860. The use of a combination of a slot and a hole allows the proximal arm 864 to move relative to the distal arm 860 as the low profile cutter blade 800 is encircled by the cutter sheath and thus constrained to move away from the shape shown in FIGS. 8A-8D. As the low profile cutter blade 800 changes shape, the curvatures in transitional sections 870 changes. FIG. 8C is a side view of low profile cutter blade 800 and FIG. 8D is a top view of the low profile cutter blade 800.

Two rivets 874 are added to retain the flush relationship between the distal arm 860 and the proximal arm 864. After the rivets 874 are pressed, the rivets 874 are made flush with the surface of the distal arm 860 and with the surface of the proximal arm 864 (lower side of rivets not visible in this view). The tip 848 as shown here does not have a cutting edge but is rounded or beveled.

While there are significant differences in the cutter blades 500, 1500 and 800, they share common characteristics that allow them to be used interchangeably with a cutter body as described in more detail below.

Details of a Cutter

FIG. 9 shows a cutter 600 which is shown in its major components. As used in the art, a cutter 600 is a cutter body 610 with a cutter blade 453 installed. While it is useful to show a particular cutter blade for purposes of FIG. 9, this is not a limitation on the type of cutter blade that may be used. If the cutter blades are made to be interchangeable with one another for a particular cutter body, the cutter blade could be of any one of a range of blade types including those described in connection with FIGS. 6-8 and could be any one of a range of cutter throw lengths or cutter blade angles.

The components of the cutter 600 identified in FIG. 9 include: a cutter rod and handle subassembly 620, cutter sheath 640, sheath liner 652 and cutter blade 453 (not to scale). The cutter sheath 640 may be made as one unitary component, but the cutter sheath 640 will often be a sub-assembly with several components. FIG. 9 has a sheath 644 and sheath grip 648 which may be connected to the sheath using a pin, set screw, adhesive; epoxy, weld, spring-loaded bayonet lock, or other connection. The sheath grip 648 may have been formed (such as molded) with the sheath as one component. The sheath grip 648 may be formed (e.g., machined or molded) from a suitable material, such as stainless steel, or medical grade high impact polymers (such as glass-filled polyethersulfone; polyphenylsulfone, e.g., RADEL®, Solvay, Inc., Houston, Tex.) that will withstand multiple sterilization cycles. As the sheath grip 648 is intended to be gripped by the user, it may have finger engaging grooves or other features to make it easy to reliably grip the sheath grip, even when wearing surgical gloves which may have fluids on them.

The cutter sheath 640 may also include a sheath liner 652 that fits within the distal end of the sheath 644 to provide a surface that makes contact with the cutter blade 453 as the cutter sheath 640 is advanced and retracted to sheathe and unsheathe the cutter blade 453.

The cutter rod and handle subassembly 620 includes the cutter rod 624 and cutter handle 628. The cutter handle 628 may be ergonomically angled, for example, from between about 90 degrees and about 180 degrees, often at about 110 degrees, relative to the base of the cutter handle 628 and a plane that is parallel to the long axis of the cutter rod 624.

More detailed description of components in the cutter rod and handle subassembly 620 are provided in connection with more detailed drawings discussed below. While the dimensions of the components will be impacted by the specific therapeutic procedures (what part of the body, needs for strength, and other attributes), for context it is useful to know that cutter rods for the trans-sacral use described herein may be in the range of about 4 millimeters to about 12 millimeters in diameter and from about 10 to about 18 inches in length.

FIGS. 10A-10C illustrate one method of connecting a cutter blade 453 to a cutter rod 410. FIG. 10A shows the distal end of a cutter body 610, including cutter rod 624, with post 632 in cutter rod slot 636. FIG. 10A also shows the distal end of the sheath liner 652, and the sheath 644.

FIG. 10A shows, the longitudinal portion 406 of the cutter blade 453 which may be placed over post 632 (also called a pin) in cutter rod 624 so that the post 632 passes through a corresponding cutter blade hole 407 (partially visible here) on the inner leg 440 of the longitudinal portion 406 of the cutter blade 453 and into a cutter blade slot 427 on the outer leg 444 of the longitudinal portion 406 of the cutter blade 453.

The cutter rod slot 636 is dimensioned to accommodate a cutter blade 453. The width of the cutter rod slot 636 is approximately the same as the width of the longitudinal portion 406 of the cutter blade 453. The curvature 642 at the distal end of the cutter blade slot 636 between the cutter rod extensions 680 (also called goal posts) accommodate the curvature of the cutter blade 453 between the longitudinal portion 406 and the portion of the cutter blade that may be extended 402 (also known as the cutter blade arm 402) (which defines the reach or throw of the cutter blade 453). The cutter rod slot 636 provides torsional support to the cutter blade arm 402 while the curvature 642 at the distal end of the cutter blade slot 636 provides axial support to the cutter blade arm 402 to work in conjunction with cutter blade edge geometries to reinforce the cutter blade 453.

The cutter rod extensions 680 provide additional support to the cutter blade 453 to reduce the tendency of the cutter blade to flex when rotated into tissue.

As cutter blade hole 407 is pinned to the cutter blade rod 624, the cutter blade 453 is affixed to the cutter blade rod 624. The cutter blade slot 427 allows some relative motion of the slotted portion of the upper leg 444 of the longitudinal portion 406 of the cutter blade 453 relative to the pinned portion of the lower leg 440 of the longitudinal portion 406 to move with the change of shape of the cutter blade 453 as it goes from sheathed to unsheathed and back to sheathed.

The portion of the cutter rod 624 that must be exposed to receive or release a cutter blade 453 may be called the engagement zone 690.

FIG. 10B shows a cutter blade 453 positioned within the exposed engagement zone 690 of a cutter rod 624.

FIG. 1C shows the same cutter rod 624 and engaged cutter blade 453 after the cutter sheath 640 (FIG. 9) visible here as sheath 644 and sheath liner 652, has been moved relative to the distal tip of the cutter rod 624 to sheathe at least a portion of the engagement zone 690 so that the pinned cutter blade 653 may not be removed until the engagement zone is sufficiently unsheathed.

To summarize one aspect of FIGS. 10A-C, a cutter blade 453 retention system retains reversibly engaged cutter blade 453 in an unsheathed position for disrupting tissue. This cutter blade retention system relies upon: A) the cutter rod slot 636 in the cutter rod 624 for receiving a longitudinal portion 406 of the cutter blade 453; B) a post oriented radially outward from the longitudinal axis of the cutter rod 624 and located within the cutter rod slot 636; and C) a sheath component (in this instance a sheath 644 and sheath liner 652) that precludes the longitudinal portion 406 of the cutter blade 453 positioned in the cutter rod slot 636 and reversibly engaged with the post 632 from becoming disengaged from the post 632 as long as the sheath component (652 and 644) is in a blade retaining position.

The slot in the cutter rod 624 may be oriented so that the cutter handle 628 (FIG. 9) is aligned with the blade arm 402 (when unsheathed). While not required, this relationship between the handle 628 and blade arm 402 is a useful way to allow the surgeon to keep track of the position of the unsheathed blade arm 402 by knowing rotational position of the cutter handle 628. Alternatively the blade arm 402 could be 180 degrees offset from the handle 628 or some other predictable relationship with the handle. While a cutter blade 453 meeting resistance within a patient's body while disrupting tissue may be stressed and not totally aligned with the handle 628, the deviation from total alignment is not of consequence as the surgeon merely needs a reasonable estimate of the current position of the blade arm 402.

One can appreciate that a surgeon would prefer to have a level of confidence that the cutter sheath was limited in its movement so that a cutter blade engaged with a cutter body did not become disengaged from the cutter body while inserted inside a patient. Thus, it is advantageous for the travel of the cutter sheath to be limited so that the engagement zone 690 is not sufficiently exposed to allow an engaged cutter blade to become disengaged.

Pivoting Dual Limit Sheath Limiter.

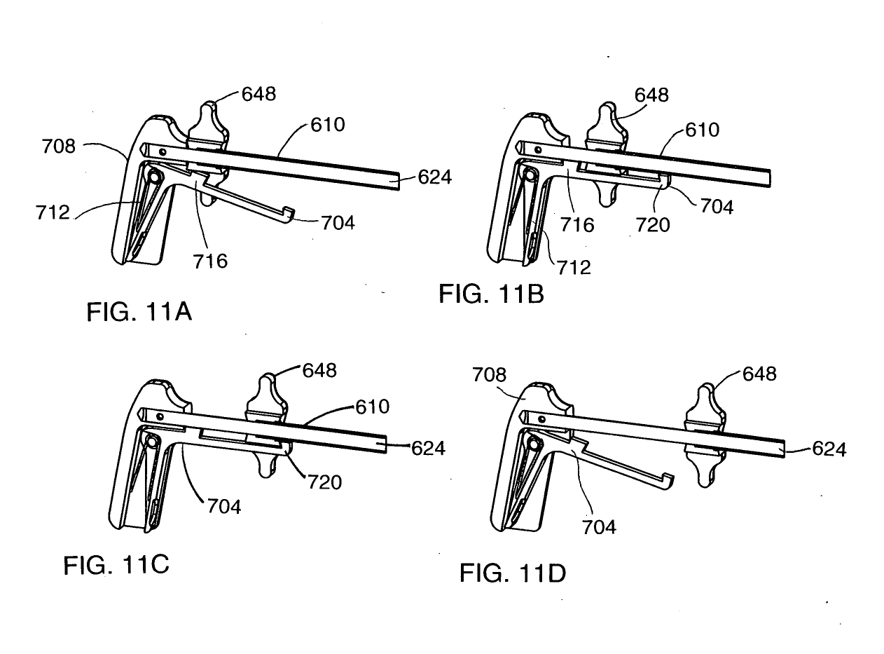

One solution to the need for a way to limit the travel of the cutter sheath is shown in FIGS. 11A-11D. To facilitate the explanation of the operation, the handle 704 is shown without some components in order to show the relevant operation.

Starting with FIG. 11A, the sheath limiter 704 is depressed within the confines of handle 708 and against the biasing force of torsion springs 712 (only the top spring is visible). As shown in FIG. 11A, the sheath limiter 704 is in the blade change position such that the cutter sheath 610 including sheath grip 648 is allowed to move into proximity to handle 704 as the back stop 716 has been pivoted out of the way. As discussed in connection with FIGS. 10A-10C, movement of the cutter sheath 610 to an extreme proximal position with respect to the handle exposes a sufficient amount of the engagement zone 690 (FIG. 10B) that a cutter blade may be engaged or disengaged with the cutter rod 624.

While the biasing force is shown here as a pair of torsion springs which may be made from stainless steel or another material, other choices are possible including a v-shaped handle spring fabricated from Nitinol™ or another shape memory alloy. Considering the moment arm and degree of angular displacement one of skill in the art will recognize that a number of other position biasing forces could be used instead of a torsion spring. In one implementation, the torsion spring rate constant ranges from between about 0.005 in-lbs/degree and about 0.132 in-lbs/degree. In another implementation, the spring rate constant ranges from between about 0.3 in-lbs/degree, to about 0.8 in-lbs/degree, and often about 0.45 in-lbs/degree.

FIG. 11B shows the cutter after the cutter sheath 610 has been advanced, likely through use of sheath grip 648 to at least partially sheathe the engagement zone 690 (FIG. 10B). Releasing the sheath limiter 704 allows the torsion springs 712 to place the sheath limiter 704 in proximity to the sheath grip 648 in what can be called an operational position. Interaction between the sheath grip 648 and the sheath limiter 704 limits the range of motion for the cutter sheath 610.

The sheath grip 648 is shown in FIG. 1B between the back stop 716 and the forward stop 720 as the sheath grip 648 has just been advanced from the blade change position. When the sheath grip 648 is abutting against the back stop 716, the cutter sheath is withdrawn relative to the distal end of the cutter body 624 thus unsheathing the cutter blade 453 to assume its unconstrained position with the blade arm 402 extended radially outward so that the cutter blade 453 is in position for use to disrupt tissue.

FIG. 11C shows the sheath grip 648 up against the forward stop 720. When the sheath grip 648 is up against the front stop 720, the cutter sheath 610 is advanced to the most distal operating position. In this position, the distal end of the cutter sheath 610 sheathes the cutter blade 453. As discussed above, a sheathed cutter blade does not extend radially beyond the cutter sheath 610 and the cutter may be moved into or out of an access path (such as an axial channel) without having contact between the cutter blade 453 and any portion of the access path.

FIG. 11D shows the sheath limiter 704 pivoted to move the forward stop 720 out of the way so that the sheath grip 648 may be advanced towards the distal end of the cutter rod 624. This may be called the disassembly position. Typically, this would be done after the cutter blade 453 was removed from the cutter rod 624. Movement of the sheath grip 648 as shown in FIG. 1D would occur during the disassembly of the cutter or cutter body 610 as the cutter sheath 640 is advanced to the point where the sheath grip 648 at the proximal end of the cutter sheath 640 comes off the distal end of the cutter rod 624.

One of skill in the art will recognize that the view shown in FIG. 11D as part of the disassembly of the cutter or cutter body could in fact be part of the process of the assembly of the cutter body as the sheath grip 648 is advanced towards the handle 708 to assume the blade change position shown in FIG. 11A. Note that the same action to pivot the sheath limiter 704 to the blade change position also places the sheath limiter 704 in the disassembly position.

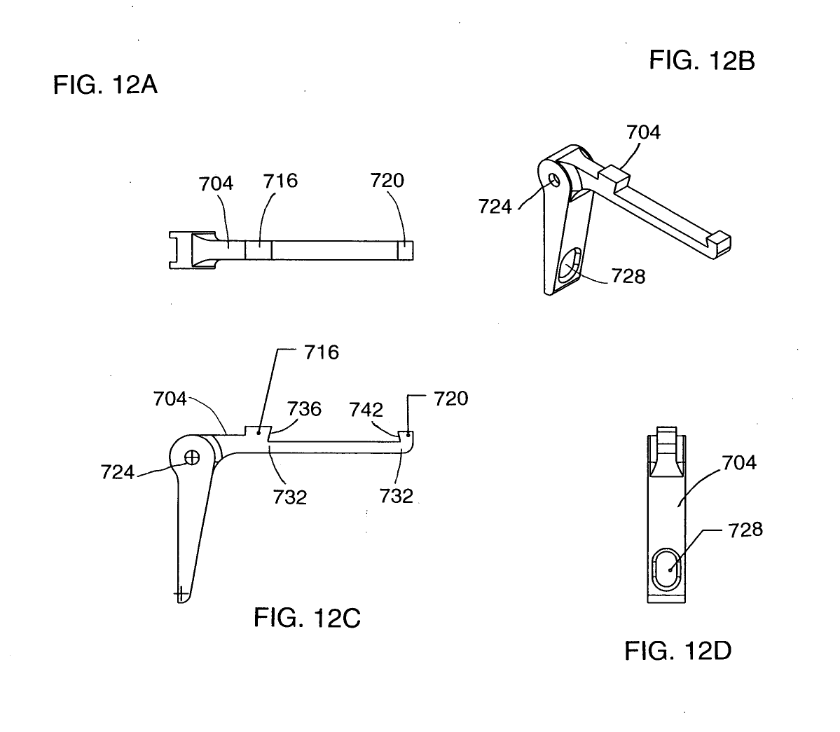

FIG. 12A-12D provides four views of the sheath limiter 704. FIG. 12A is a top view of a sheath limiter 704 and shows the back stop 716 and forward stop 720. FIG. 12 B is a top perspective view of the sheath limiter 704 and includes the pivot axis 724. The sheath limiter 704 as shown here has an optional indentation 728 for finger placement so that a user can easily find the portion of the handle to depress to pivot the sheath limiter 704.

FIG. 12C is a side view of the sheath limiter 704 and shows the forward stop 720 and back stop 716. Note that from this view it can been seen that the sheath limiter 704 has an anti-slip feature 732. This anti-slip feature 732 is used on both the forward and back stops but could be used on only one of the two. By having a recessed portion of the relevant sheath limiter faces 736 and 742 and having corresponding projections on the sheath grip 648, the potential for the sheath limiter 704 to accidentally rotate open is reduced. More specifically, the engagement of the sheath grip 648 when adjacent to the forward stop 720 (when the cutter blade is sheathed for transport) resists accidental opening as the sheath grip 648 would need to be moved back towards the handle before the sheath limiter 704 could be pivoted to move to the disassembly position discussed above.

Likewise, when the sheath grip 648 is against the back stop 716 (when the cutter blade is exposed for use in cutting), the sheath grip 648 needs to be moved away from the back stop 716 in order to pivot the sheath limiter 704 and move the back stop 716 out of the way so that the cutter body 610 may assume the blade change position with the engagement zone 690 sufficiently exposed to allow removal of an engaged cutter blade 453. One of skill in the art can envision other forms of engagement between the sheath grip (or whatever portion of the cutter sheath engages with the sheath limiter) and the relevant faces of the sheath limiter so that it is less likely that a sheath limiter in the operational position could accidentally rotate to either a blade change position or a disassembly position.

FIG. 12D provides a front view of the sheath limiter 704 including optional indentation 728.

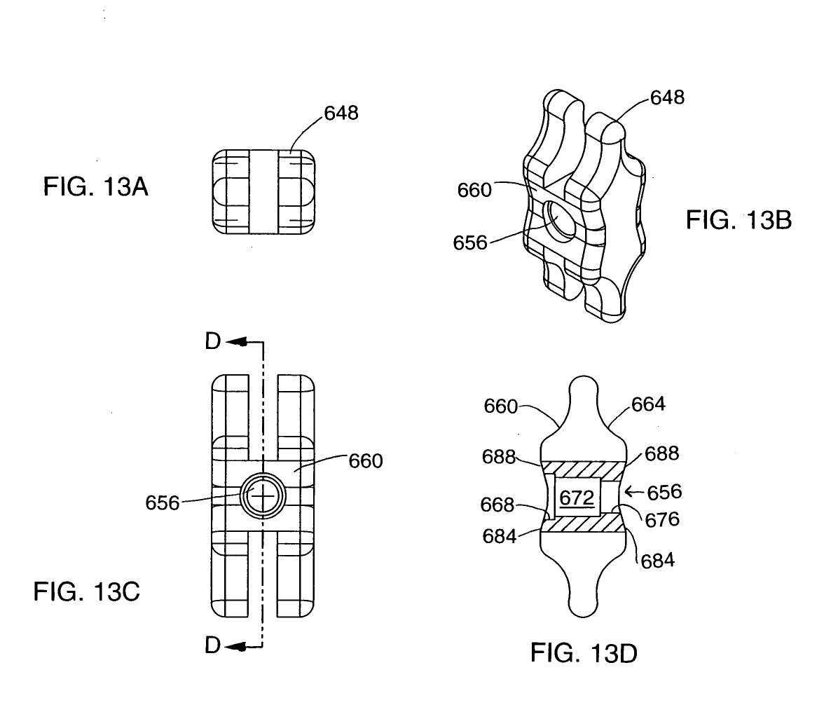

FIG. 13A-13D shows four views of a sheath grip 648. FIG. 13A is a top view. FIG. 13B is a top perspective view looking at the bore 656 on the distal side of the sheath grip 648 which shows the oversized bore 668 that may be used if the sheath grip 648 is welded to the sheath. FIG. 13C is a view looking at the into the series of bore diameters 676, 672, and 668 (best seen in FIG. 13D). FIG. 13D is a cross section of FIG. 13C taken along section line D-D showing the three bore diameters. Bore diameter 676 allows the sheath grip 648 to slide along the cutter rod 624. Bore diameter 672 can receive the proximal end of the sheath 644. Bore diameter 668 is slightly larger than bore diameter 672 and may be used if the sheath grip 648 is welded to the sheath 644. Sloped surfaces 684 can interact with anti-slip feature 732 on sheath limiter 704. Optionally, the sheath grip 648 may be made in a symmetric manner so that it can be flipped over so that sloped surfaces 688 engage with the sheath grip 648. This removes one possible source of error when assembling the cutter body 610.

Details on Sheath Liner.

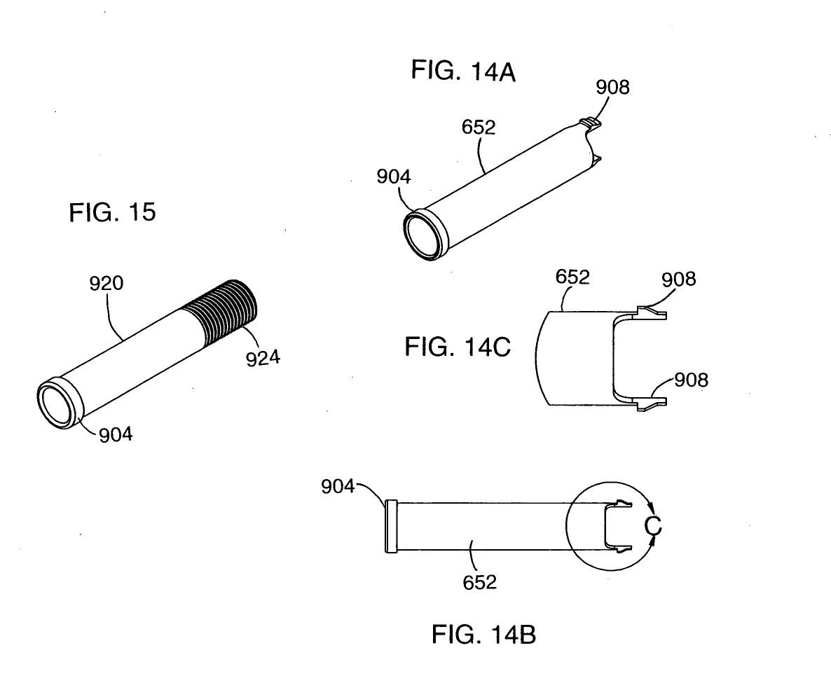

As shown in FIG. 9 and FIG. 10A, a cutter sheath may be designed to accommodate a removable sheath liner 652. The sheath 644 may have snap lock gaps 670 as indicated on FIG. 9. FIG. 14A-C shows three views of a sheath liner 652 as shown in FIG. 9. FIG. 14A shows a top perspective view showing the distal ring 904 visible in FIG. 10A as it protects the distal tip of the sheath 644 from abrasion from the cutter blade 453 as the sheath is moved to constrain or release from constraint the cutter blade 453. FIG. 14B provides a top view of the sheath liner 652 showing the pair of snap locks 908 that engage with snap lock gaps 670 in sheath 644. FIG. 14C provides an enlarged view of the snap locks 908 showing the region C in FIG. 14B.

The sheath liner 652 may provide the advantage of reducing the coefficient of friction between the cutter blade 453 and the distal end of the sheath 644. The cutter blade 453 may be formed from a shape memory alloy including a nickel-titanium shape memory alloy such as Nitinol™. The cutter sheath 644 may be made from an appropriate grade of stainless steel. To reduce the friction between the cutter blade 453 and the inner surface of the cutter sheath 644, the sheath liner 652 may be made of a material with a coefficient of friction with the Nitinol™ cutter blade that is lower than the coefficient of friction between Nitinol™ and stainless steel. If the sheath liner 652 is to be reused in a procedure for another patient, the material for the sheath liner 652 may be chosen for the material's ability to withstand multiple sterilization cycles (which may include temperatures in excess of 130 degrees Centigrade). Ultra-high molecular weight polyethylene (UHMWPE) is one such material. Other material choices include poly-tetrafluoroethylene (PTFE) or PTFE-loaded polymers, e.g., DELRIN® (acetyl copolymer available from E.I. dupont de Nemours, Inc., Wilmington, Del.) or fabrication from a another biocompatible material with a relatively low coefficient of friction (e.g., lower than that of a stainless steel sheath and a blade fabricated from Nitinol™), that is sufficiently durable to withstand multiple re-blading events and preferably will withstand a plurality of sterilization cycles (for example, fabrication from certain polysulfones; polyvinylidene fluorides (PVDF)).

Additional material choices for the sheath liner that will be suitable for receiving machined threads include polysulfone (Udel), polyphenylsulfone (Radel R), polyamide 6/12 (Nylon 6/12), & polyetherimine (Ultem 1000), and polyvinylidene fluoride (PVDF) and its copolymers such as Kynar® (Elf Atochem North America).

Alternatively, the sheath liner 652 may be fabricated from a material without an advantageous coefficient of friction, but merely to afford the option of replacing this part of the cutter sheath 640 as this portion may become worn. Thus, in some uses it may be reasonable to have the sheath liner made from the same material as the sheath 644. The sheath liner shown in FIG. 15 may be preferred to that shown in FIG. 14 when working with stainless steel or other materials that do not lend themselves to use with snap locks.

FIG. 15 shows a perspective view from the distal end of the sheath liner 920 which has distal ring 904 as described above but a set of external threads 924 to engage a corresponding set of female threads (not shown) in the interior of a threaded sheath (not shown).

The sheath liners of any shape may be modified by adding a dry lubrication such as poly-tetrafluoroethylene (PTFE). Favorable surface characteristics may be added to sheath liners by using an electroless nickel plating treatment. Optionally, the surface of a sheath liner after electroless nickel plating can receive a coating of poly-tetrafluoroethylene (PTFE) to reduce the coefficient of friction. Other processes may be used to reduce the coefficient of friction between the relevant surfaces of the sheath liner and the cutter blade. Examples of such processes may include electropolishing or bead blasting

Sheath liners may be fabricated for a single use. In this context a use is utilization during one or more surgical procedures for a patient with perhaps multiple blade changes but not a subsequent surgical procedure for another patient after a sterilization cycle. Other sheath liners may be designed for many uses and thus need to tolerate many sterilization cycles. For example, a sheath liner may have an intended life of 50 uses. If the sheath liner is being used to extend the useful life of the sheath, then the sheath should be able to tolerate many more sterilization cycles than the replaceable sheath liner. The various cutter body components with the exception of the sheath liner, may be designed to have the same useful life so that the cutter body (other than the replaceable sheath liner) does not normally need replacement parts other than the sheath liner.

The use of a sheath liner of any type, while potentially advantageous is optional. A sheath could be designed without a sheath liner. The sheath could receive a surface treatment such as a dry lubrication such as poly-tetrafluoroethylene (PTFE) may be used, or the sheath may receive an electroless nickel plating treatment with or without the additional PTFE coating.

Thumb Screw Implementation

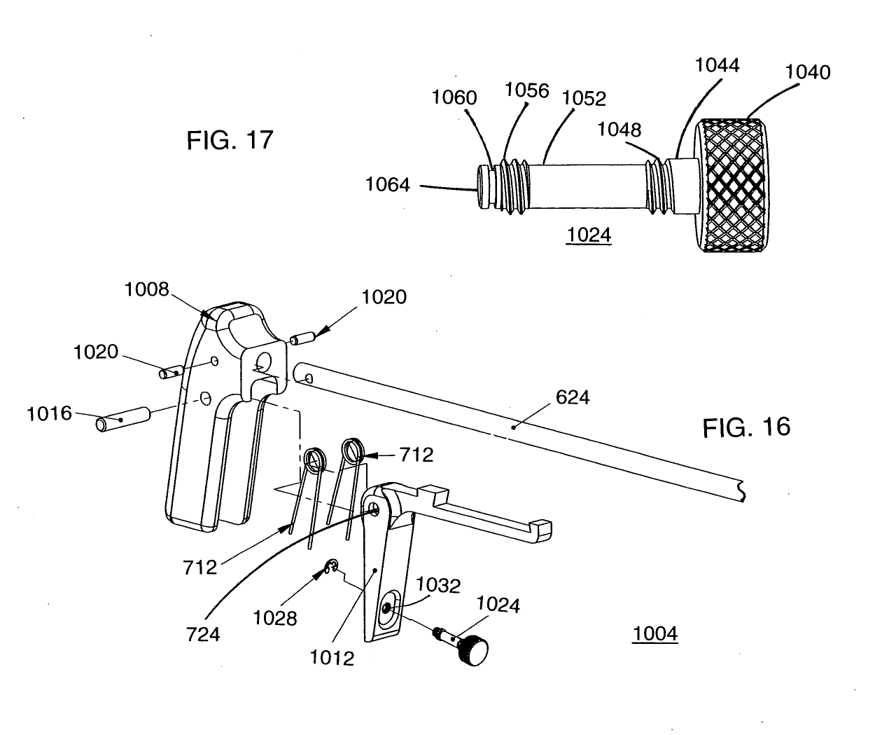

Another implementation using a sheath limiter similar to that shown above is shown in FIG. 16. More specifically, FIG. 16 is an exploded diagram of the proximal end of a cutter rod and handle assembly 1004 showing only a portion of the cutter rod 624. The other components are cutter handle 1008, sheath limiter 1012 with threaded bore 1032; dowel pin 1016 that passes through pivot axis 724, a pair of torsion springs 712, a pair of dowel pins 1020 that are used to retain the cutter rod 624, a dual threaded thumb screw 1024, and a retaining ring 1028.

Dual threaded thumb screw 1024 is shown in greater detail in FIG. 17. Starting from the distal section of the dual threaded thumb screw 1024, there is knurled section 1040, shoulder 1044, distal threaded section 1048, unthreaded section 1052, proximal threaded section 1056, groove 1060, and proximal end 1064.

The operation of the sheath limiter 1012 with dual threaded thumb screw 1024 is very similar to the sequence of steps described in connection with FIG. 11. However, the dual threaded thumb screw 1024 provides an extra protection against inadvertently shifting the sheath limiter 1012 into a non-operational position (either a blade loading position where a loaded bladed could become disengaged, or a disassembly position where the cutter sheath could come off the distal end of the cutter rod 624). The extra protection comes from the use of the dual threaded thumb screw 1024 which can be positioned into either an operational position or an extraordinary position.

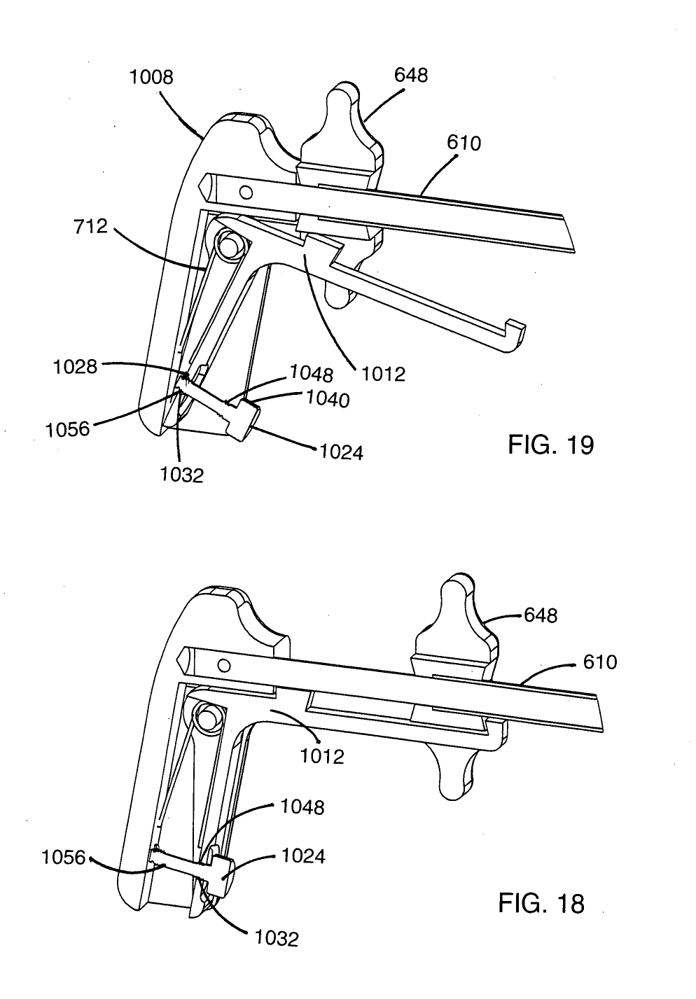

FIG. 18 shows a portion of a cross section of cutter rod and handle sub assembly 1004 in the operational position. In the operational position, the distal threaded section 1048 of dual threaded thumb screw 1024 are engaged with corresponding threads on the threaded bore 1032 of sheath limiter 1012. When the threads are engaged in this way, the proximal end 1064 of the dual threaded thumb screw 1024 is in contact with the rear wall of the cutter handle 1008 which prevents the sheath limiter 1012 from pivoting to any position other than the operational position. In the operational position, the cutter sheath 610 may be moved by moving the sheath grip 628 to selectively sheathe and unsheathe any engaged cutter blade without sufficiently exposing the engagement zone 690 (FIG. 10B) to allow an engaged cutter blade 453 to become disengaged.

FIG. 19 shows a portion of a cross section of a cutter rod and handle sub assembly 1004 in the blade exchange position, one of the extraordinary as opposed to operational positions. Note that in order to enable the sheath limiter 1012 to overcome the bias of the torsion springs 712 (only one spring visible in FIG. 19), the knurled section of the dual threaded thumb screw 1024 was rotated to disengage the distal threaded section 1048 from the threaded bore 1032 and the dual threaded thumb screw 1024 was pulled out so that the proximal threaded section 1056 could be engaged with threaded bore 1032. Retaining ring 1028 positioned in groove 1060 (See FIG. 17) retains the dual threaded thumb screw 1024 from being screwed out of the assembly altogether.

With the dual threaded thumb screw 1024 in the extended, extraordinary position, the sheath limiter 1024 may be rotated to overcome the torsion springs 712 and allow the cutter body to assume a blade change position. While the sheath limiter 1024 will pivot back to an operational position if allowed to do so, anyone holding the cutter handle 1008 will notice that the dual threaded thumb screw 1024 is in a position that enables extraordinary travel of the cutter sheath 610.

The process of moving the dual threaded thumb screw 1024 back to the operational position is the reverse of the process for moving it from the operational position to the extraordinary position. As the sheath limiter 1012 must be moved out of the operational position to an extraordinary position to allow for disassembly of the cutter body, the same process would be used to move the dual threaded thumb screw 1024 to the extraordinary position in order to enable the disassembly.

Another type of Sheath Limiter

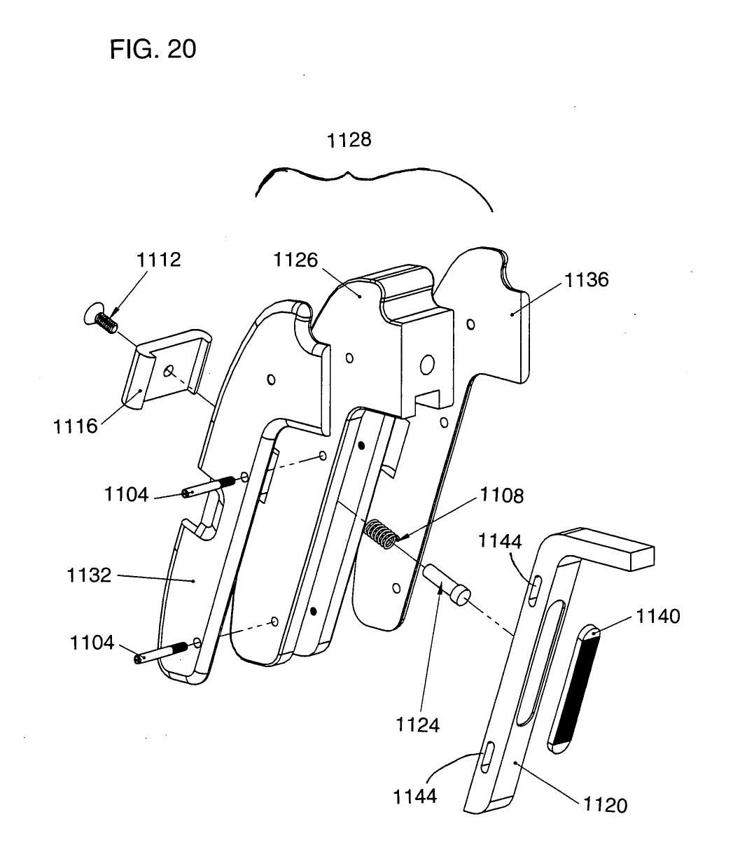

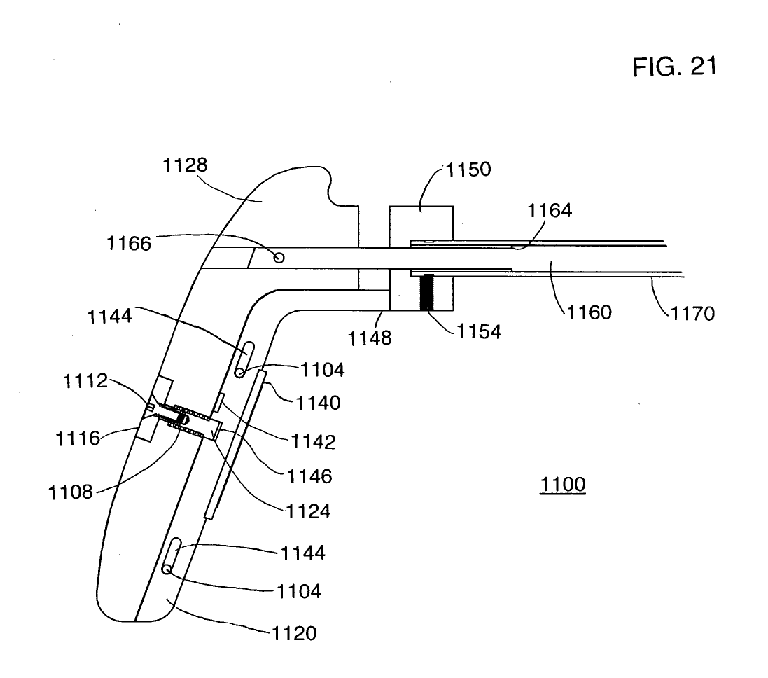

FIG. 20 shows an exploded view of a cutter handle and sheath limiter from a cutter body 1100. After an overview of the components, the operation of the sheath limiter will be explained in the context of FIGS. 21-23.

The components are: two slotted headless screws 1104, a compression spring 1108, position screw 1112, pull knob 1116 (sometimes called a spring retainer knob), sheath limiter 1120 with slots 1144 to allow slotted movement of sheath limiter 1120 with respect to slotted headless screws 1104, spring retainer 1124 (which has a threaded bore that engages the external threads on the position screw 1112), offset T-handle 1126, right handle grip 1132, left handle grip 1136, (collectively referenced as handle 1128) and sheath limiter grip 1140. The sheath limiter grip 1140 may be fabricated from, silicone rubber or from other suitable, sterilizable materials, to provide a soft surface to improve comfort and also to facilitate the surgeon's effective use. The relationship of the various components is conveyed by the exploding diagram and need not be repeated here.

FIG. 21 shows the sheath limiter 1120 in the operational position and the cutter body 1100 positioned to have a cutter blade 453 (not shown here) unsheathed but retained so that the cutter may be used to disrupt tissue.

The sheath grip 1150 is connected to the sheath 1170 by a set screw 1154 but it could be connected by other types of connection including a bayonet type spring lock, welding, adhesives, or other forms of connection. The cutter rod 1160 has stepped shoulder 1164 that is clearly visible here and will be discussed below.

Note that sheath grip 1150 is abutting the back stop 1148 on the sheath limiter 1120. The back stop 1148 imposes a limit on the sheath grip 1150 and thus on the sheath 1170. This back stop prevents the sheath 1170 from sufficiently exposing the engagement zone 690 (not shown here) to allow an engaged cutter blade 453 to become disengaged. The sheath limiter 1120 is capable of slotted movement over slotted headless screws 1104 in slots 1144 but this slotted movement is prohibited by the operational position of the pull knob 1116, position screw 1112, compression spring 1108, and spring retainer 1124 which are collectively engaged with operational bore 1146 (as opposed to the shallower blade change bore 1142). Note that the cutter rod 1160 may be retained in the handle 1128 by a connector 1166 such as a pin or a headless screw.

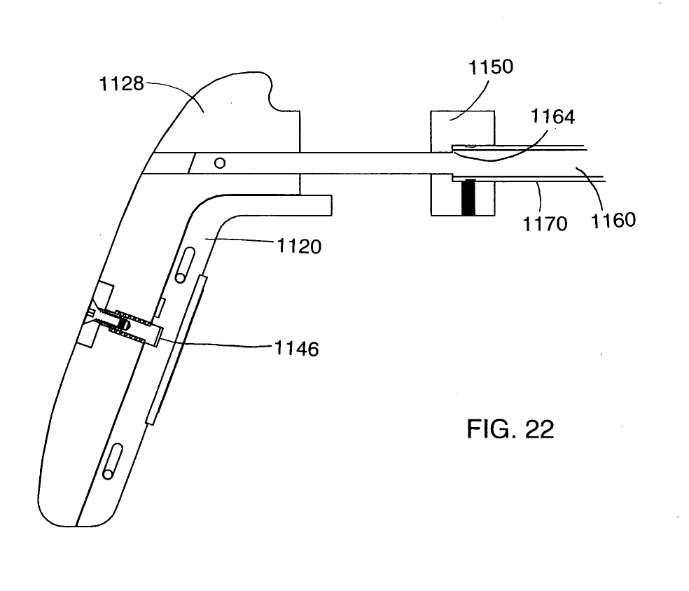

FIG. 22 is very much like FIG. 21 in that the sheath limiter 1120 is still in the operational position with the operational bore 1146 engaged and the sheath limiter 1120 precluded from slotted movement. Sheath grip 1150 has been moved away from handle 1128 towards the distal end of the cutter rod 1120 but has reached a forward stop as the sheath grip 1150 has made contact with the stepped shoulder 1164. In this position, an engaged cutter blade 453 would be sheathed and ready for transport.

Alternatively, a set screw, pin other connection implement that connect the sheath grip 1150 to the sheath 1170 could protrude radially inward such that the tip of the set screw engages with the shoulder 1164 so that contact between the pin and the shoulder 1164 stops the distal movement of the cutter sheath.

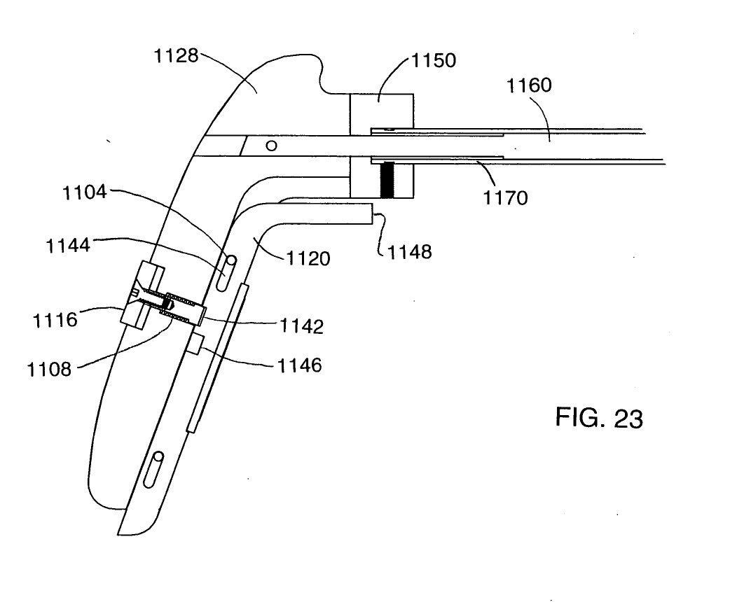

FIG. 23 shows the sheath limiter 1120 outside of the operational position and in the position for a blade change. The sheath limiter 1120 has moved downward to place the slotted headless screws 1104 at the top of the slots 1144. The movement of the sheath limiter 1120 has moved the back stop 1148 out of the way of the sheath grip 1150. Likewise the sheath grip 1150 is abutting the handle 1128 and thus sufficiently exposing the engagement zone 690 to allow an engaged cutter blade to be removed and replaced.

The steps to attain the positioning shown in FIG. 23 from a start in an operational position are as follows.

Pull the pull knob 1116 away from the handle 1128 to pull spring retainer 1124 out of the operational bore 1146 so that the sheath limiter 1120 may move downward (away from the cutter rod 1160). Pulling will be in resistance to the compression spring 1108. The compression spring 1108. In one implementation used the spring rate ranges from between about 0.5 inches per pound (commonly represented as in/lb) and 50 in/lb, and often between about 5-6 in/lb.

After the sheath limiter 1120 has begun to move, release the pull knob 1116.

Continued movement of the sheath limiter 1120 downward to at or near the end of the allowable slotted travel will cause the spring biased spring retainer 1124 to engage with the shallow blade change bore 1142. As the blade change bore 1142 is shallower than the operational bore 1146, the pull knob 1116 will project outward from the handle 1128 to provide a visual and tactile indicator that the sheath latch 1120 is in a position to allow the sheath to expose the engagement zone. As it would not be desirable to insert a cutter into a patient before the sheath limiter 1120 is returned to its operational position, this raised pull knob 1116 provides an important reminder.

Once the sheath limiter 1120 is in the blade change position, the sheath grip 1150 may be moved to abut the handle 1128 as shown in FIG. 23 to allow the removal and replacement of the cutter blade.

To reverse the process, the sheath grip 1150 is moved to the operational range, then the pull knob 1116 is pulled away from the handle to disengage the spring retainer 1124 from the blade change bore 1142.

The sheath limiter 1120 may be pushed upward and the pull knob 1116 released. As the sheath limiter 1120 comes to near the end of its slotted travel, the spring retainer 1124 becomes engaged with the deeper operational bore 1146, allowing the pull knob 1116 to seat as shown in FIGS. 21 and 22.

As this latch limiter 1120 does not have a forward stop, the latch limiter 1120 does not need to be placed into a special position in order to disassemble the cutter body 1120. Disassembly may include removing the connector 1166 to allow the cutter rod 1160 to be removed from the handle 1128. Note that while the cutter rod 1160 is placed into a bore that passes through the entire handle 1128, the handle could be modified to have a blind bore that passes partway from the distal end of the handle to the proximal end of the handle and thus is closed on the proximal end of the handle.

One of ordinary skill in the art could combine the forward and back limits of the pivot based latch limiter and the corresponding sheath grip with the latch limiter motion and locking mechanism of the latch limiter shown in FIGS. 20-23 to have a different implementation that did not need a stepped shoulder for a forward stop.

Note that the sheath limiter of the type shown in FIG. 20 could be implemented using a rivet in place of the position screw 1112.

Materials Choices and Other Details

In the context of the present disclosure, the term “biocompatible” refers to an absence of chronic inflammation response or cytotoxicity when or if physiological tissues are in contact with, or exposed to (e.g., wear debris) the materials and devices of the present disclosure. In addition to biocompatibility, in another aspect of the present disclosure it is preferred that the materials comprising the instruments are sterilizable; visible and/or imageable, e.g., fluoroscopically.

Components used the cutter body and cutter blades described above are configured and constructed (e.g., cannulated; solid; blunt; beveled; angled; retractable; fixed; tilted; axially aligned; offset; extendible; exchangeable; stiff; flexible; deformable; recoverable; removable; biocompatible; able to be sterilized and machined; moldable; reusable; disposable) in accordance with optimal intended function and in deference to biomechanical and safety constraints (e.g., designed to withstand wear and breakage).

The cutter rod and cutter sheath are typically fabricated from a metal or metal alloy, for example 316 stainless steel for the sheath and 17-4 alloy for the cutter rod. Other materials may be used provided that they provide the necessary characteristics. Depending on the material selected, these components could be either machined or injection molded.

More specifically, the cutter rods, sheaths, and handles may be fabricated from heat treated stainless steel alloys, such as those described in ASTM F899-02 Standard Specifications for Stainless Steels for Surgical Instruments or, for example, 17-4 alloy where torque or wear resistance may be a consideration. Alternatively, components may be formed (machined, following heat treatment of blank rod), from high tensile strength (greater than substantially from about 250K-300K psi), high fatigue strength metal alloy rod, not containing Fe, such as, titanium alloys (e.g., Ti6Al4V); cobalt chrome super alloy; or MP35N rod (ultrahigh tensile strength [265K psi; 34.K ksi modulus of elasticity; 11.7K ksi shear modulus] non-magnetic, Ni—Co—Cr—Mo alloy available from Carpenter Technology Corporation, Reading Pa.) according to the biomechanical properties being selected by design (e.g., substantially modulus matched; or where, for instance, the need for superior fatigue strength is indicated).

Also alternatively, sheaths may be fabricated by molding a polymer including those fabricated from medical grade PVDF such as Kynar®; polyether-ether-ketone (PEEK) such as that commercially available from Invibio Inc., in Lancashire, United Kingdom, or polyether-ketone-ketone (PEKK) available from Coors-Tech Corporation, in Colorado, or alternatively, conventional polymethylmethacrylate (PMMA); ultra high molecular weight polyethylene (UHMWPE), or other suitable polymers (e.g., into which threads are able to be machined).

The cutter handle (or various components of a multi-component handle) may be fabricated (such as molded or machined) from, a range of materials including: stainless steel; hardened/anodized aluminum, or a suitable high strength medical grade polymer, such as a glass filled polyphenysulfone (e.g., RADEL®).

As discussed in more detail in U.S. patent application Ser. No. 11/712,548 filed Feb. 28, 2007 for Cutter for Preparing Intervertebral Disc Space and application Ser. No. 11/712,241 for Specialized Cutter Blades for Preparing Intervertebral Disc Space reference above, the cutter blades can be formed from strip material that is preferably a shape memory alloy in its super-elastic or austenitic phase at room and body temperature and that ranges in width from about 0.10 inches (2.5 mm) to about 0.20 inches (5 mm) and in thickness from about 0.015 inches (0.38 mm) to about 0.050 inches (1.3 mm). Cutter blades may be formed that are generally able to be flexed in excess of 100 cycles without significant shape loss, and twisted up to one and ½ full turns (about 540 degrees) without breakage. This is twisting of one end of the cutter blade relative to another portion of the cutter blade.

The shape memory feature is useful in allowing the cutter blade to resume the extended position which is in shape memory when the cutter blade is unsheathed and thus unconstrained. The shape memory feature is also useful in helping the cutter blade to resume its intended shape after being distorted while being rotated within the intervertebral disc space and receiving uneven resistance to motion.

In one implementation, the cutting blade and cutter blade edge is formed from a super-elastic, shape memory metal alloy that preferably exhibits biocompatibility and substantial shape recovery when strained to 12%. One known suitable material that approximates the preferred biomechanical specifications for cutter blades and cutter blade edges and blade arms is an alloy of nickel and titanium (e.g., Ni₅₆—T₄₅ and other alloying elements, by weight), such as, for example, Nitinol strip material #SE508, available from Nitinol Devices and Components, Inc. in Fremont, Calif. This material exhibits substantially full shape recovery (i.e., recovered elongation when strained from about 6%-10%, which is substantially better than the recovered elongation at these strain levels of stainless steel).

The shape and length of the formed cutter blade in general varies for the different cutting modes. The shape memory material can be formed into the desired cutter blade configuration by means of pinning alloy material to a special forming fixture, followed by a heat-set, time-temperature process, as follows: placing the Nitinol strip (with the blade's cutting edge(s) already ground) into the forming fixture and secured with bolts; and placing the entire fixture into the oven at a temperature ranging from about 500° C. to about 550° C. (e.g., where optimum temperature for one fixture is about 525° C.) for a time ranging from between about 15 to about 40 minutes (e.g., where the optimum time for one fixture is about 20 minutes). Flexible cutter blades formed from Nitinol in this manner are particularly suited for retraction into a shaft sleeve, and are able to be extended to a right angle into the disc space. Moreover, they are able to mechanically withstand a large number of cutting “cycles” before failure would occur.

The cutting blade edges are preferably ground with accuracy and reproducibly. The angle of the inclined surface of the blade relative to the blade's flat side surface typically ranges from about 5 degrees to about 70 degrees, often about 20 degrees to about 50 degrees. Thus, the blade edge angle may be approximately 30 degrees relative to the blade's side surface.

The cutter blades may be configured with serrations are formed by a wire EDM (Electrical Discharge Machining) process to optimize design profiles. For higher manufacturing volumes, cutter blades are formed via profile grinding; progressive die stamping; machining, or conventional EDM.

As will be understood by one of skill in the art, certain components or sub-assemblies of the assemblies of the present disclosure may alternatively be fabricated from suitable (e.g., biocompatible; sterilizable) polymeric materials, and, for example, may be coated (e.g., with PTFE) to reduce friction, where appropriate or necessary.

For example, the cutter sheath can be fabricated from polymeric material, stainless steel, or a combination of stainless steel tubing with a low friction polymeric sleeve such as UHMWPE, HDPE, PVDF, PTFE loaded polymer. The cutter sheath typically has an outer diameter (O.D.) of about 0.31 inches (7 mm) to about 0.35 inches (9 mm).

Another way to decrease the coefficient of friction between the cutter blade and the sheath liner (or sheath is used without a liner) is to apply a biocompatible coating such as a surfactant or hydrophilic hydrogel, or the like. The cutter blade arm may be lubricated, the sheath liner (or sheath) may be lubricated or both may be lubricated.

Alternatives

Cutter rods may be specialized to work with specific cutter blades with specific blade angles. For example, it may be advantageous to use a cutter rod for a 45 degree blade that allows the 45 degree blade to begin its downward angle while still in contact with the cutter rod. Alternatively, a standard cutter rod could be used for a range of cutter blade angles and the variation in blade angles would be handled in the cutter blades after the cutter blade has left contact with the cutter rod. A combination of both strategies might call for a few different cutter rods such as a 45 degree cutter rod and a 90 degree cutter rod and using attributes of the cutter blades to provide an expanded range of cutter blade angles.

Additional Limiter Stops.

While the sheath limiters shown above had either just a back stop or a back stop and a forward stop, a sheath limiter may have more than two stops. For example, a sheath limiter may have a sheathed forward stop and a sheathed back stop so that a sheathed cutter blade being inserted into a patient's body that receives resistance from a portion of the patient's body has the sheath limiter sheathed back stop to assist in maintaining the sheath in the distal position as the sheathed back stop prevents the sheath from moving towards the handle and thus partially unsheathing the sheathed cutter blade while the cutter blade is in transit towards the place for tissue disruption. Effectively the sheath grip would be sandwiched between the sheathed forward stop and the sheathed back stop so that there was no significant range of motion possible for the sheath until the sheathed back stop was removed. In most instances the spring force of the sheathed cutter blade will be ample for maintaining the position of the sheath to keep the cutter blade fully sheathed, but this option exists.

Another instance where having an additional sheath limiter stop may be useful is for surgeons using a cutter blade with a 45 degree blade angle (See FIG. 5 and representative blade angle 464). A surgeon may wish to use the cutter blade at something different than 45 degrees such as approximately 60 degrees or 90 degrees. Advancing the sheath grip from the unsheathed operational position to a partially sheathed position would cause the distal end of the sheath to alter the blade angle upward from 45 towards 60 degrees or 90 degrees depending on the amount that the sheath is extended. Rather than having the surgeon maintain pressure on the sheath grip against the spring force of the cutter blade that attempts to return to the original blade angle of 45 degrees, it may be appreciated by the surgeon to have an option of using an intermediate stop to hold the sheath in this partially advanced position.

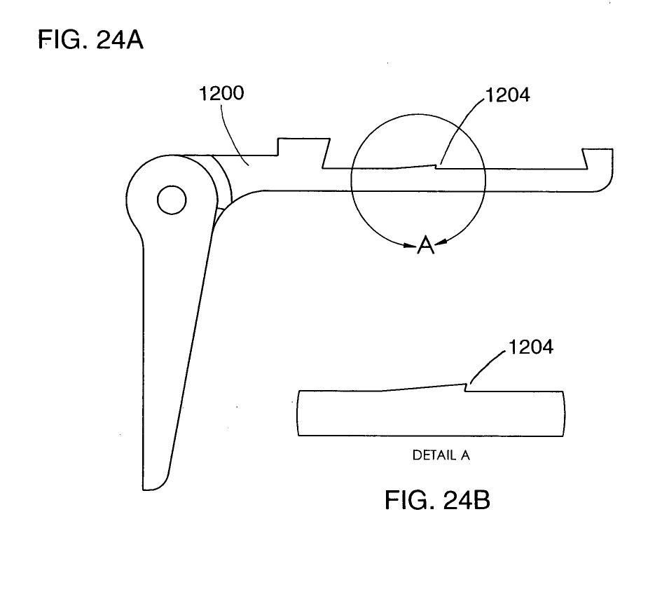

FIGS. 24A-24B illustrate a sheath limiter 1200 with an intermediate stop 1204. The intermediate stop 1204 may be a reduced height stop so that a minor movement of the sheath limiter actuator insufficient to allow the sheath grip to move past either the back stop of the forward stop is sufficient to clear this intermediate stop. This type of minor movement may be allowed in a system that uses the dual threaded thumb screw (1024 in FIGS. 17-19) without having to move the dual threaded thumb screw to a position to allow movement past the forward or back stop. Looking at FIG. 24B, it is evident that the handle grip may move from the proximal side of the intermediate stop to the distal side of the intermediate stop without any special user interaction in implementations adapted for this function, but moving up and over the intermediate stop to go from the distal side to the proximal side would require some user intervention.

While FIGS. 24A-24B show a singe intermediate stop, one could implement two or more intermediate stops, if that was useful in the context of a particular cutter and if the portion of the cutter sheath that engages the sheath limiter is adapted to allow two stops to be placed within proximity of one another while allowing the portion of the cutter sheath to seat properly. For example, there may be a practical limit on how many intermediate stops could be placed on sheath limiter that engages the sheath grip based on the dimensions of the sheath grip.

Limiter does not have to Engage the Grip

While the examples given have used the sheath grip as the portion of the sheath that interacts with the sheath limiter, this is not a requirement. While it may be efficient to use the sheath grip as both a grip and as the point of engagement with the sheath limiter, these two functions could be separated. If separated, the sheath limiter engagement could occur somewhere more distal than the sheath grip.

Alternative Configuration of Longitudinal Portion of Cutter Blade

While the closed loop cutter blades disclosed above have used a cutter blade hole 407 on the inner leg 440 that is the longitudinal portion placed against the cutter rod 410 and a cutter blade slot 427 on the outer leg 444 that is the longitudinal portion not placed against the cutter rod 410, one of skill in the art will appreciate that one could modify the cutter blades and the cutter rod to allow the use of the cutter blade hole on the upper leg and the cutter blade slot on the lower leg without deviating from the spirit of the teachings of the present disclosure.

Likewise, one could modify the cutter blades shown above to allow for at least some types of cutter blades with holes on both the upper and lower legs of the longitudinal portion so that once pinned, there was not relative motion of one leg relative to the other leg. An implementation lacking the opportunity for relative motion of the two legs would rely more on the ability of the shape memory material to resume a given shape as the pinned longitudinal portions could not move relative to one another to help with the transformation.

Other Way to Induce Relative Movement

The text associated with FIGS. 10A-10C described how an engagement zone 690 could be selectively exposed and partially sheathed to either allow the removal and insertion of cutter blades 453 or to prevent an engaged blade from being disengaged. Particular ways to allow or disallow such movement were demonstrated with respect to a sheath that moves relative to the cutter handle to sheathe or unsheathe the distal end of the cutter-rod 624.

One of ordinary skill in the art will recognize that relative motion of the distal end of the sheath relative to the distal end of the cutter rod could be achieved by moving the sheath relative to a point of reference (such as the handle) and holding the cutter rod fixed with respect to that same point of reference, or by moving the cutter rod relative to the point of reference and keeping the sheath fixed with respect to that point of reference. (The third case of moving both with respect to the point of reference and to each other would be combination of the other two sources of relative motion).

To implement the same retaining system as described above, one could have a cutter with a cutter blade that fits into a cutter rod with a cutter blade engagement zone. This cutter would not have a cutter rod and handle sub-assembly but would have a cutter sheath and handle sub-assembly including: a handle; a cutter sheath; and a rod limiter. The rod limiter would be adapted to engage and disengage from a portion of the cutter rod (such as a cutter rod grip) so that the portion of the cutter rod assembly may be constrained to move axially between a back stop and a forward stop.

The rod limiter may be implemented in manners similar to that shown above and may be placed on either the cutter blade side of the handle or on the user side of the handle.

The cutter rod limiter would allow the cutter rod to push forward to hyperextend beyond the operational range, the distal portion of the cutter rod beyond the sheath to sufficiently expose the engagement zone to allow for a blade exchange. Once the cutter rod was pulled back to an operational position with the cutter blade retained, the rod limiter would limit the travel range of the cutter rod to allow it to come back to sheathe the cutter blade for transport or push forward to unsheathe the cutter blade to allow it to be used to disrupt tissue.

Depending on the specifics of how the cutter rod was assembled into a cutter body, there may be a rod limiter position that allows the cutter rod to be removed from the cutter body.

As noted above, there may be additional intermediate stops to hold the cutter rod in a position that presses the sheath against a portion of the cutter blade to change the blade angle. One of skill in the art will recognize that due to the reversal of motion needed to generate this effect, the cutter rod would be pulled back into the sheath to achieve this effect and thus would need to be limited in the ability to move out, away from the handle in order to maintain the intermediate position that causes a change in blade angle.

Advancement and Retraction Achieved by Methods Other than Push/Pull

One of skill in the art will recognize that the relative motion imposed by pulling and pushing a grip to move a component could be implemented by other mechanisms such as threaded engagement to advance or retract the component to be moved. While the examples given above implied that a user would move the grip relative to the handle, the inventive concepts could be implemented using power or power assisted tools including electrical, pneumatic, or hydraulic based systems.

Other Procedures.

While the focus of this disclosure has been on the use of cutter blades for tissue disruption and any tissue extraction of disrupted tissue, the reusable cutter bodies and the cutter blades described above may also be used in conjunction with other methods, including hydro-excision; laser; and other to perform partial or complete nucleectomies, or to facilitate other tissues' manipulation (such as fragmentation and or extraction).

The cutters described above have been described in the context of use within an intervertebral disc space. One of skill in the art will recognize that the desirable attributes of the disclosed cutters could be used within other medical procedures that access material to be disrupted (most likely for removal before a subsequent therapeutic procedure or to harvest material for use in a therapeutic procedure) by delivery of a cutter blade in a sheathed state through a lumen before the cutter blade assumes an unsheathed (extended) position in which the cutter blade has as a shape memory. One of skill in the art will recognize that the dimensions of the cutter blade and related components may need to be adjusted to meet the relevant anatomic dimensions and the dimension of the lumen used for providing access. The dimensions and properties of cutter blades will be contingent on their intended use. Cutter blades intended for cutting bone will have characteristics that are different from cutter blades for use on softer forms of tissue. While there may not be cartilage covered vertebral body endplates to preserve or scrape (depending on the desired results) there may be other anatomic structures that need to be protected from cutting edges or alternatively need to be scraped as part of site preparation, thus making many of the specific teachings of the present disclosure relevant.

Kits

Various combinations of the tools and devices described above may be provided in the form of kits, so that all of the tools desirable for performing a particular procedure will be available in a single package. Kits in accordance with the present disclosure may include preparation kits for the desired treatment zone, i.e., provided with the tools necessary for disc preparation. Disc preparation kits may differ, depending upon whether the procedure is intended to be in preparation for therapy of one or more vertebral levels or motion segments. The disc preparation kit may include a plurality of cutters and cutter blades. For example for a single level fusion kit, anywhere from 3 to 7 cutter blades may be provided. In a two level fusion kit, anywhere from 5 to 14 cutter blades may be provided. The set of cutter blades will likely include an assortment of cutter blades. The assortment of cutter blades is likely to be impacted by the specific procedure to be performed (such as fusion versus mobility preservation) and possibly based on the patient anatomy (which may impact the range of cutter blade throw lengths and the cutter blade angles needed). In addition to having a variety of blade angles and throw lengths, a kit may contain cutter blades of more than one type such as a mix of closed loop cutter blades and low profile cutter blades. The kit may contain cutter blades with different cutter blade edges, such as including serrated blades and non-serrated blades.

Typically, a kit will include cutter blades that include a small radial cutter blade, a medium radial cutter blade, and a large radial cutter blade. The kit will typically also include small, medium, and large cutter blades with a blade angle of 45 degrees. Kits for specific procedures may include other cutter assemblies with specific cutter blades for specific uses for example inclusion of cutter blades chosen for there ability to cut into and cause bleeding in either the inferior or superior endplates. All of the cutters blades are one-time (one patient) use then disposable. Certain other components comprised within the cutter body may be disposable or reusable.

The disc preparation kit may (optionally) additionally include one or more tissue extraction tools, for removing fragments of the nucleus. In a one level kit, 3 to 8 tissue extraction tools, perhaps 6 tissue extraction tools are provided. In a two level disc preparation kit, anywhere from about to 8 to about 14 tissue extraction tools, perhaps 12 tissue extraction tools are provided. The tissue extraction tools may be disposable.

Teachings May be Used in Isolation or Combined

One of skill in the art will recognize that some of the alternative implementations set forth above are not universally mutually exclusive and that in some cases additional implementations can be created that employ aspects of two or more of the variations described above. Additional variations may be created by implementing some but not all of the teachings provided for a particular implementation provided above. Likewise, the present disclosure is not limited to the specific examples provided to promote understanding of the various teachings of the present disclosure. Moreover, the scope of the claims which follow covers the range of variations, modifications, and substitutes for the components described herein as would be known to those of skill in the art.

What is claimed is:

1. A cutter for disrupting tissue, the cutter comprising:
a cutter blade;
a cutter sheath; and
a cutter rod and handle sub-assembly including:
a handle;
a cutter rod with a distal end that is distal from the handle;
wherein the cutter blade is adapted to engage the cutter rod at an engagement zone on the perimeter of the cutter rod near the distal end of the cutter rod such that a portion of the cutter blade extends beyond the distal end of the cutter rod when the cutter blade is constrained by the cutter sheath;
the cutter sheath adapted to allow a proximal end of the cutter sheath to pass over the distal end of the cutter rod to engage a sheath limiter that limits movement of the cutter sheath to a range of movement along a long axis of the cutter rod.

2. The cutter of claim 1 wherein the sheath limiter includes:
a forward stop to limit the movement of the proximal end of the cutter sheath from moving towards the distal end of the cutter rod; and
a back stop to limit the movement of the proximal end of the cutter sheath from moving towards the handle.

3. The cutter of claim 1 wherein the sheath limiter is adapted to limit the movement of a sheath grip and thus limit the movement of the cutter sheath.

4. The cutter of claim 1 wherein the cutter sheath has a sheath grip and has a separate component other than the sheath grip for engagement with the sheath limiter.

5. The cutter of claim 2 wherein the sheath limiter has an anti-slip feature on the forward stop such that a cutter sheath engaged with the forward stop must be moved along the long axis towards the handle before the sheath limiter may be disengaged to allow the cutter sheath to move distal of the forward stop.

6. The cutter of claim 5 wherein the anti-slip feature is a recessed portion of a sheath limiter face that at least partially engages with a corresponding surface on the cutter sheath to prevent the sheath limiter from disengaging from the cutter sheath when the corresponding surface is in contact with the face.

7. The cutter of claim 2 wherein the sheath limiter has an anti-slip feature on the back stop such that a cutter sheath engaged with the back stop must be moved along the long axis away from the handle before the sheath limiter may be disengaged to allow the cutter sheath to move beyond the back stop towards the handle to expose an engagement zone of the cutter rod.

8. The cutter of claim 7 wherein the anti-slip feature is a recessed portion of a sheath limiter face that at least partially engages with a corresponding surface on the cutter sheath to prevent the sheath limiter from disengaging from the cutter sheath when the corresponding surface is in contact with the sheath limiter face.

9. The cutter of claim 2 wherein the sheath limiter has a third stop between the forward stop and the back stop.

10. The cutter of claim 2 wherein the sheath limiter has a third stop between the forward stop and the back stop and this third stop maintains the cutter sheath at the limit imposed by the forward stop.

11. The cutter of claim 2 wherein the sheath limiter includes a biasing means to bias the cutter against non-intended movement of the proximal end of the cutter sheath beyond the forward stop.

12. The cutter of claim 11 wherein the biasing means involves elastic deformation of at least one component.

13. The cutter of claim 11 wherein the biasing means includes a threaded engagement between the biasing means and the sheath limiter.

14. The cutter of claim 2 wherein the engagement zone is at least partially obstructed to prevent loading a cutter blade on the cutter rod when the sheath limiter is limiting the movement of the proximal end of the cutter sheath between the forward stop and the back stop.

15. The cutter rod of claim 14 wherein a cutter blade can become engaged with the cutter rod when the sheath limiter is manipulated to allow the proximal end of the cutter sheath to move beyond the back stop towards the handle to expose the engagement zone of the cutter rod.

16. The cutter of claim 1 wherein the cutter blade is made from a shape memory material such that the cutter assumes one shape when constrained by the cutter sheath and assumes a second shape when the cutter sheath is moved away from the distal end of the cutter rod.

17. The cutter of claim 1 wherein the cutter blade engages a post on the cutter rod.

18. The cutter of claim 1 wherein the cutter sheath includes:
a sheath; and
a sheath grip affixed to the proximal end of the sheath.

19. The cutter of claim 1 wherein the cutter sheath includes:
a sheath;
a sheath grip affixed to the sheath; and
a sheath liner at the distal end of the sheath whereby a worn sheath liner may be replaced without replacing the sheath.

20. The cutter of claim 19 wherein the sheath liner is at least partially fabricated from a biocompatible metal.

21. The cutter of claim 1 wherein the cutter sheath includes:
at least one component that has a hydrophilic coating.

22. The cutter of claim 1 wherein the cutter sheath includes at least one component that has a electroless nickel surface treatment.

23. The cutter of claim 22 wherein the surface treatment is an electroless nickel surface treatment with a polytetrafluoroethylene coating.

24. The cutter of claim 1 wherein the cutter sheath includes at least one component that has a polytetrafluoroethylene coating.

25. The cutter of claim 1 wherein the cutter sheath includes at least one component that has received a processing to reduce friction between the component and the cutter blade.

26. The cutter of claim 1 wherein the cutter sheath includes:
a sheath;
a sheath grip affixed to the sheath; and
a sheath liner at the distal end of the sheath, the sheath liner selected from a material that has a coefficient of friction between the cutter blade and the sheath liner that is less than the coefficient of friction between the cutter blade and a material used for the distal end of the sheath.

27. The cutter of claim 26 wherein the sheath liner engages a set of threads within the sheath.

28. The cutter of claim 26 wherein the sheath liner has a snap-lock engagement with the sheath.

29. The cutter of claim 26 wherein the sheath liner when engaged has a distal portion of the sheath liner that is distal to a distal end of the sheath such that the distal end of the sheath liner is the most distal portion of the cutter sheath.

30. A cutter for disrupting tissue, the cutter comprising:
a cutter blade;
a cutter sheath; and
a cutter rod and handle sub-assembly including:
a handle;
a cutter rod with:
a distal-end that is distal from the handle; and
a cutter blade engagement zone; and
a sheath limiter, the sheath limiter adapted to engage and disengage from a portion of the cutter sheath so that the portion of the cutter sheath assembly may be constrained to move axially between a back stop and a forward stop;
the cutter sheath adapted to interact with a sheath limiter to operate in at least two positions:
a blade change position exposing the cutter rod blade engagement zone by allowing movement of the portion of the cutter sheath beyond a sheath limiter back stop towards the handle to allow engagement or disengagement of a cutter blade with the cutter rod; and
an operational position limiting the movement of the portion of the cutter sheath within a limited range between the back stop and forward stop where the range is sufficient to allow a cutter body with an engaged cutter blade to have the cutter blade sheathed for transport and selectively unsheathed to allow the engaged cutter blade to assume a cutting position without becoming disengaged from the cutter rod.

31. The cutter of claim 30 wherein the cutter sheath is adapted to interact with a sheath limiter to operate in a disassembly position that allows the portion of the cutter sheath to move beyond the forward stop to allow the disengagement of the cutter sheath from the cutter rod and handle sub-assembly.

32. The cutter of claim 30 wherein the portion of the cutter sheath is a portion of a sheath grip.

33. The cutter of claim 30 wherein the portion of the cutter sheath is located at a proximal end of the cutter sheath.

34. The cutter of claim 30 wherein the disassembly position of the sheath limiter allows the portion of the cutter sheath to move from distal of the forward stop to proximal of the forward stop during assembly of the cutter.

35. The cutter of claim 30 wherein the manipulation to place the sheath limiter in the disassembly position is sufficient to place the sheath limiter in the blade change position such that a sheath limiter in a disassembly position will allow the portion of a cutter sheath to move from the distal end of the cutter rod past the forward stop and past the back stop.

36. The cutter of claim 30 wherein the sheath limiter is associated with a pivot such that at least a portion of the sheath limiter rotates in an arc relative to a long axis of the cutter rod to move from the operational position to the blade change position.

37. The cutter of claim 30 wherein at least a portion of the sheath limiter translates relative to the long axis of the cutter rod to move from the operational position to the blade change position.

38. A cutter for disrupting tissue, the cutter comprising:
a cutter blade;
a cutter sheath; and
a cutter rod and handle sub-assembly including:
a handle;
a cutter rod with:
a distal end that is distal from the handle; and
a cutter blade engagement zone; and
a sheath limiter, with a forward stop, an intermediate stop, and a back stop;
the cutter sheath adapted to interact with a sheath limiter to operate the cutter in at least four modes:
a blade change mode exposing the cutter rod blade engagement zone by allowing movement of the portion of the cutter sheath beyond a sheath limiter back stop towards the handle to allow engagement or disengagement of a cutter blade with the cutter rod;
a first cutting mode involving the back stop with the cutter blade assuming a first cutting position;
a second cutting mode involving the intermediate stop with the cutter blade assuming a second cutting position different from the first cutting position; and
a sheathed mode involving the forward stop that sheathes the cutter blade for transport.

39. The cutter of claim 38 wherein the cutter sheath adapted to interact with a sheath limiter to operate the cutter in a fifth mode that allows the relative motion of the cutter sheath to the cutter rod to separate the cutter sheath from the cutter rod as part of disassembly of the cutter.

40. A cutter for disrupting tissue, the cutter comprising:
a cutter blade;
a cutter rod with:
a distal end that is distal from the handle; and
a cutter blade engagement zone;
a cutter sheath and handle sub-assembly including:
a handle;
a cutter sheath with a distal end that is distal from the handle; and
a rod limiter, the rod limiter adapted to engage and disengage from a portion of the cutter rod so that the portion of the cutter rod assembly may be constrained to move axially between a back stop and a forward stop;
the cutter rod adapted to interact with a rod limiter to operate in:
a blade change position exposing the cutter rod blade engagement zone by allowing movement of the portion of the cutter rod beyond a rod limiter forward stop towards the distal end of the cutter sheath to allow engagement or disengagement of a cutter blade with the cutter rod; and
at least one cutting position with the engaged cutter blade at least partially unsheathed without becoming disengaged from the cutter rod; and
a sheathed position with the cutter blade sheathed for transport.

41. The cutter of claim 40 wherein the cutter rod is adapted to interact with the rod limiter to operate in a disassembly position which allows the cutter rod to be disengaged from the cutter sheath and handle sub-assembly.

42. The cutter of claim 40 wherein the portion of the cutter rod that engages with the rod limiter is associated with a cutter rod grip.

43. The cutter of claim 40 wherein the rod limiter is associated with a pivot such that at least a portion of the rod limiter rotates in an arc relative to a long axis of the cutter sheath to move from a cutting position to the blade change position.

44. The cutter of claim 40 wherein at least a portion of the rod limiter translates relative to the long axis of the cutter sheath to move from a cutting position to the blade change position.

45. A cutter, for disrupting tissue, the cutter comprising:
a cutter blade;
a cutter sheath; and
a cutter rod and handle sub-assembly including:
a handle;
a cutter rod with:
a distal end that is distal from the handle; and
a cutter blade engagement zone; and
a sheath limiter, the sheath limiter adapted to selectively serve as a back stop to limit the movement of the cutter sheath towards the handle;
the cutter sheath adapted to limit the forward movement of the cutter sheath towards the distal end of the cutter rod by interaction with a forward stop associated with the cutter rod and handle sub-assembly but independent of the sheath limiter;
the assembled cutter adapted to operate in at least three modes:
a blade change mode exposing the cutter rod blade engagement zone by allowing movement of the portion of the cutter sheath beyond a sheath limiter back stop towards the handle to allow engagement or disengagement of a cutter blade with the cutter rod;
a first cutting mode involving the back stop with the cutter blade assuming a first cutting position; and
a sheathed mode involving the forward stop that sheathes the cutter blade for transport.

46. The cutter of claim 45 wherein the assembled cutter is adapted to operate in a second cutting mode involving an intermediate stop with the cutter blade assuming a second cutting position different from the first cutting position.

47. The cutter of claim 45 wherein the cutter sheath is a single component.

48. The cutter of claim 45 wherein the cutter sheath is a sub-assembly with at least two different components.

49. The clutter of claim 48 wherein the cutter sheath includes a sheath liner which is a replaceable component.

50. The cutter of claim 45 wherein the cutter moves to the to the blade change mode from the first cutting mode by movement of the sheath limiter from a sheath limiting position to a retracted position.

51. The cutter of claim 50 wherein the sheath limiter may be secured in the retracted position.

52. The cutter of claim 51 wherein the sheath limiter may be secured in the sheath limited position

53. The cutter of claim 51 wherein a user holding the handle of the cutter would be able to receive input to the palm of the user to indicate that the sheath limiter was secured in the retracted position.

54. The cutter of claim 45 wherein the cutter blade engagement zone is positioned relative to the handle so that a hand grip portion of the handle has a first radial orientation with respect to the long axis of the cutter rod and an unstressed radially extended cutter blade in the cutting position also has the first radial orientation with respect to the long axis of the cutter rod such that a user of the cutter may estimate the current radial orientation of the radially extended cutter blade in the cutting position within a patient's body based on the current radial position of the hand grip portion of the handle.

55. The cutter of claim 45 wherein the cutter blade engagement zone is positioned relative to the handle so that a hand grip portion of the handle has a first radial orientation with respect to the long axis of the cutter rod and an unstressed radially extended cutter blade in the cutting position has a known relationship with the first radial orientation with respect to the long axis of the cutter rod such that a user of the cutter may estimate a current radial orientation of the radially extended cutter blade in the cutting position within a patient's body based on the current radial position of the hand grip portion of the handle.

56. The cutter of claim 45 wherein the forward stop is implemented using a cutter rod with a stepped shaft.

57. A cutter for disrupting tissue, the cutter comprising:
a cutter blade;
a cutter body, the cutter body comprising:
a cutter sheath; and
a cutter rod and handle sub-assembly including:
a handle;
a cutter rod with a distal end that is distal from the handle;
wherein:
the cutter blade is adapted to engage the cutter rod at an engagement zone on the perimeter of the cutter rod near the distal end of the cutter rod such that an engaged cutter blade may be retained by movement of the cutter sheath relative to the engagement zone; and
while the cutter blade is retained, the cutter blade may be selectively sheathed for transport and selectively unsheathed for use to disrupt tissue.

58. The cutter of claim 57 wherein the cutter sheath is a single component.

59. The cutter of claim 57 wherein the cutter sheath is a sub-assembly with more than one component.

60. The cutter of claim 57 wherein subsequent movement of the cutter sheath relative to the engagement zone allows for disengagement of the engaged cutter blade.

61. The cutter of claim 60 wherein the cutter sheath is adapted to interact with a sheath limiter,
the sheath limiter, when in a first position, limiting movement of the cutter sheath to a range of movement along a long axis of the cutter rod such that the engaged cutter blade remains engaged with the cutter rod, and
the sheath limiter, when in a second position, allowing the movement of the cutter sheath to expose the engagement zone and allow for the engagement or disengagement of the cutter blade.

62. The cutter of claim 61 wherein the sheath limiter in the first position has a forward stop and abutting a portion of the cutter sheath against the forward stop places an engaged cutter blade in a sheathed position for transport.

63. The cutter of claim 62 wherein the sheath limiter in the first position has a back stop and abutting the portion of the cutter sheath against the back stop partially unsheathes an engaged cutter blade for use to disrupt tissue.

64. The cutter of claim 63 wherein the sheath limiter has an intermediate stop between the back stop and the forward stop.

65. The cutter of claim 64 wherein the intermediate stop maintains the portion of the cutter sheath in proximity to the forward stop to help maintain the engaged cutter blade in the sheathed position for transport.

66. The cutter of claim 64 wherein abutting the portion of the cutter sheath against the intermediate stop sheathes a portion of the cutter blade to alter the blade angle of the cutter blade.

67. The cutter of claim 57 wherein the unsheathed cutter blade extends out radially from the long axis of the cutter rod and current radial orientation of the handle with respect to the long axis of the cutter rod indicates the approximate radial orientation of the unsheathed cutter blade with respect to the long axis of the cutter rod.

68. The cutter of claim 57 wherein the radial orientation of a grip portion of the handle is aligned with the radial orientation of the unsheathed cutter blade with respect to the long axis of the cutter rod.

69. A cutter blade retention system to retain a reversibly engaged cutter blade in a position for disrupting tissue, the cutter blade retention system comprising:
a cutter rod slot in a cutter rod for receiving a longitudinal portion of the cutter blade;
a post oriented radially outward from the longitudinal axis of the cutter rod and located within the cutter rod slot; and
a sheath that precludes the longitudinal portion of the cutter blade positioned in the cutter rod slot and reversibly engaged with the post from becoming disengaged from the post as long as the sheath is an blade retaining position;

70. The cutter blade retention system of claim 69 wherein the cutter blade retention system prevents rotation and axial movement of the longitudinal portion of the retained cutter blade.

71. The cutter blade retention system of claim 69 wherein the width of the cutter rod slot is close to the width of the longitudinal portion of the cutter blade in order to provide torsional support to the cutter blade while disrupting tissue.

72. The cutter blade retention system of claim 69 wherein a curved portion of the cutter rod slot at the distal end of the cutter rod accommodates a curved portion of a reversibly engaged cutter blade in the position for disrupting tissue.

73. The cutter blade retention system of claim 72 wherein a pair of cutter rod extensions extend beyond the curved portion of the cutter rod slot to support the cutter blade when used for disrupting tissue.

74. The cutter blade retention system of claim 69 wherein the longitudinal portion of the cutter blade has a first leg with a post hole and a second leg with a slot and the post engages the post hole and the slot to allow the second leg to move relative to the first leg while the retained reversibly engaged cutter blade transitions from a sheathed transport position to the position for disrupting tissue.

75. The cutter blade retention system of claim 74 wherein the post engages the post hole before engaging the post slot as the cutter rod slot in the cutter rod receives the longitudinal portion of the cutter blade.

76. The cutter blade retention system of claim 74 wherein the post engages the post slot before engaging the post hole as the cutter rod slot in the cutter rod receives the longitudinal portion of the cutter blade.

77. The cutter blade retention system of claim 69 wherein the longitudinal portion of the cutter blade has a first leg with a post hole and a second leg with a second post hole and the post engages the two post holes to engage the reversibly engaged cutter blade.

78. The cutter blade retention system of claim 74 wherein the sheath includes an outer sheath and a sheath liner that receives contact from the cutter blade as the retained reversibly engaged cutter blade transitions from the sheathed transport position to the position for disrupting tissue.

79. The cutter blade retention system of claim 69 wherein the sheath component may be advanced towards the distal end of the cutter rod to sheathe the cutter blade to place the cutter blade in the sheathed transport position.

80. The cutter blade retention system of claim 69 wherein the sheath component may be moved away from the distal end of the cutter rod to sufficiently unsheathe the cutter blade to allow the cutter blade to be removed from the cutter rod slot and replaced with a different cutter blade.

81. A cutter body and blade kit for disrupting tissue, the cutter body and blade kit comprising:
a cutter blade of a first type;
a cutter blade of a second type, different from the cutter blade of the first type;
a cutter body, the cutter body comprising:
a cutter sheath; and
a cutter rod and handle sub-assembly including:
a handle;
a cutter rod with a distal end that is distal from the handle;
wherein the cutter blade of the first type and the cutter blade of the second type are both adapted to engage the cutter rod at an engagement zone on the perimeter of the cutter rod near the distal end of the cutter rod such that an engaged cutter blade of the first type or the second type may be retained by movement of the cutter sheath relative to the engagement zone and while retained may be selectively sheathed for transport and selectively unsheathed for use to disrupt tissue.

82. The kit of claim 81 wherein the cutter blade of the first type forms a loop with an inner and outer perimeter.

83. The kit of claim 82 wherein the cutter blade of the first type has at least one cutting edge located on the outer perimeter of the loop adapted to abrade vertebral endplates and promote bleeding.

84. The kit of claim 82 wherein the cutter blade of the first type has all of its cutting edges on the inner perimeter of the loop so as to avoid abrading vertebral endplates.

85. The kit of claim 81 wherein the cutter blade of the first type is a thin cutter blade for use in a thin disc.

86. The kit of claim 81 wherein the cutter blade of the first type has a shorter blade throw than the cutter blade of the second type such that use of the cutter blade of the second type to disrupt tissue after using the cutter blade of the first type to disrupt tissue will result in an enlarged region of disrupted tissue.

87. The kit of claim 81 wherein the cutter blade of the first type first cutter blade angle and the cutter blade of the second type has a second type of cutter blade angle, different from the first type, such that use of the cutter blade of the second type to disrupt tissue after using the cutter blade of the first type to disrupt tissue will result in an enlarged region of disrupted tissue.

88. A cutter blade kit for use with a cutter body that receives exchangeable cutter blades to form a surgical cutter for disrupting tissue, the cutter blade kit comprising:
a cutter blade of a first type;
a cutter blade of a second type, different from the cutter blade of the first type;
wherein the cutter blade of the first type and the cutter blade of the second type are both adapted to engage a cutter rod at an engagement zone on the perimeter of the cutter rod near the distal end of the cutter rod such that an engaged cutter blade of the first type or the second type may be retained by movement of the cutter sheath relative to the engagement zone and while retained may be selectively sheathed for transport and selectively unsheathed for use to disrupt tissue.

89. The kit of claim 88 wherein the cutter blade of the first type forms a loop with an inner and outer perimeter.

90. The kit of claim 89 wherein the cutter blade of the first type has at least one cutting edge located on the outer perimeter of the loop adapted to abrade vertebral endplates and promote bleeding.

91. The kit of claim 89 wherein the cutter blade of the first type has all of its cutting edges on the inner perimeter of the loop so as to avoid abrading vertebral endplates.

92. The kit of claim 88 wherein the cutter blade of the first type is a thin cutter blade for use in a thin disc.

93. The kit of claim 88 wherein the cutter blade of the first type has a shorter blade throw than the cutter blade of the second type such that use of the cutter blade of the second type to disrupt tissue after using the cutter blade of the first type to disrupt tissue will result in an enlarged region of disrupted tissue.

94. The kit of claim 88 wherein the cutter blade of the first type first cutter blade angle and the cutter blade of the second type has a second type of cutter blade angle, different from the first type, such that use of the cutter blade of the second type to disrupt tissue after using the cutter blade of the first type to disrupt tissue will result in an enlarged region of disrupted tissue.

95. The kit of claim 88 wherein the kit includes a sheath liner for use in the distal end of the cutter sheath.

96. A method of replacing a cutter blade in a cutter for disrupting tissue, the cutter comprising a cutter blade and a cutter body, the method comprising:
moving a sheath limiter from an operational position to a blade change position;
after the sheath limiter is in the blade change position, moving a cutter sheath relative to an engagement zone on a cutter rod to expose the engagement zone;
disengaging a previously engaged first cutter blade from the engagement zone;
engaging a second cutter blade in the engagement zone previously engaged with the first cutter blade;
moving the cutter sheath relative to the engagement zone to retain the second cutter blade; and
moving the sheath limiter to the operational position to allow for movement of the sheath relative to the engagement zone that is insufficient to expose the engagement zone for disengagement of the second cutter blade.

97. The method of claim 96 wherein the first cutter blade is not the same type of cutter blade as the second cutter blade;

98. The method of claim 97 wherein the first cutter blade has a shorter blade throw than the second cutter blade such that use of the second cutter blade to disrupt tissue after using the first cutter blade to disrupt tissue will result in an enlarged region of disrupted tissue.

99. The method of claim 97 wherein the first cutter blade has a first cutter blade angle with respect to the cutter rod and the second cutter blade has a second cutter blade angle with respect to the cutter rod wherein the first cutter blade angle is different from the second cutter blade angle such that use of the second cutter blade to disrupt tissue after using the first cutter blade to disrupt tissue will result in an enlarged region of disrupted tissue.

100. The method of claim 96 further including after use of the second cutter blade and any subsequently engaged cutter blades, removing the cutter sheath from the cutter body.

101. The method of claim 100 further including removing a sheath liner from the removed cutter sheath and inserting a replacement sheath liner into the cutter sheath.

102. The method of claim 96 wherein the movement of the sheath limiter from the blade change position to the operational position includes rotating the sheath limiter with respect to the cutter rod.

103. The method of claim 96 wherein the movement of the sheath limiter from the blade change position to the operational position includes translating the sheath limiter with respect to the cutter rod.

104. The method of claim 96 wherein the sheath limiter may be reversibly affixed to the cutter body to preclude movement from the operational position to the blade change position while affixed.
